(19) United States
(12) Patent Application Publication
(10) Pub. No.: US 2025/0218591 A1
(43) Pub. Date: Jul. 3, 2025

(54) CLASSIFICATION OF INSTERSTITIAL LUNG DISEASE

(71) Applicant: UNIVERSITY OF VIRGINIA PATENT FOUNDATION, Charlottesville, VA (US)

(72) Inventor: Imre Noth, Charlottesville, VA (US)

(21) Appl. No.: 19/006,155

(22) Filed: Dec. 30, 2024

Related U.S. Application Data
(60) Provisional application No. 63/616,322, filed on Dec. 29, 2023.

(51) Int. Cl. G16H 50/20; A61B 5/00; A61B 5/08; G16H 10/60

(52) U.S. Cl. CPC G16H 50/20; A61B 5/08; A61B 5/7267; G16H 10/60

(57) ABSTRACT

Various processes, algorithms, and systems are provided herein for assisting physicians in distinguishing among related diseases, such as distinguishing connective tissue associated interstitial lung disease from idiopathic pulmonary fibrosis. Methods for generating such processes, algorithms, and systems are also disclosed. In some embodiments, a preliminary diagnosis of a set of possible diseases is obtained, along with protein count information from a patient's blood sample. Additional, patient-specific information (e.g., age, sex, etc.) may also be obtained. The data is processed by a trained machine learning algorithm, to output a differential diagnosis of which of the set of possible diseases is present for that patient. Based on the diagnosis, a treatment course can be selected, and further information can be tracked regarding the patient's outcome.

### CROSS-REFERENCE TO RELATED APPLICATION(S)

This application claims priority to U.S. Provisional Application No. 63/616,322, filed on Dec. 29, 2023, the entire content of which (including all Figures and Appendices) is incorporated herein by reference.

### STATEMENT REGARDING FEDERALLY SPONSORED RESEARCH

This invention was made with government support under UG3HL145266 awarded by the National Heart Lung and Blood Institute. The government has certain rights in the invention.

### BACKGROUND

Patients with interstitial lung disease (ILD) present with heterogeneous syndromes, requiring evaluation of clinical, radiographic, and pathologic features. Generally speaking, the term “ILD” is used to refer to a category of pulmonary disorders which may include a broad variety of diseases and syndromes. Often, ILD presents symptoms including inflammation and/or scarring (fibrosis) of the lung, typically in the lung interstitium. These disorders can be progressive (though not in all cases), and can lead to long term loss of lung function.

Among the many types of ILD disorders, two classes present symptoms that make them particularly difficult to differentiate. One class includes connective tissue disorders (CTD-ILD), which involve autoimmune mechanisms. In contrast, the other class, idiopathic pulmonary fibrosis (IPF), is a different diagnosis that requires the exclusion of autoimmune diseases, or other causes.

Both CTD-ILD and IPF often present similar symptoms, and both can lead to lung parenchymal fibrosis, often sharing a usual interstitial lung pattern on a CT and a biopsy. Due to their similar presentation and symptoms, there can be difficulty in discerning whether a given patient has either CTD-ILD or IPF. Current standards for differentiating a diagnosis as between these two diseases are cumbersome, involve input of several different physician specialties, and are surprisingly inaccurate. In many cases, there may not be consensus even among the treating specialists as to which disease a given patient has.

For example, CTD-ILD is often associated with underlying autoimmune diseases, such as rheumatoid arthritis, systemic sclerosis, Sjogren's syndrome, and mixed connective tissue disease (many of which are, themselves, sometimes difficult to diagnose). In some patients, symptoms of the underlying disease that is associated with CTD-ILD can manifest prior to or along with the ILD symptoms—but this is not always the case and is not by itself determinative. Therefore, diagnosis of CTD-ILD tends to involve use of radiologic imaging (e.g., CT scans or chest x-rays) which may show pneumonia-like presentation in the patient's lungs (non-specific interstitial pneumonia patterns are common, depending on the associated underlying disease) and/or blood tests (such as various antibody panels which can help in some circumstances, but again not all types of CTD-ILD disorders can be confirmed by blood test alone). However, both IPF and CTD-ILD can often (though not always) exhibit similar patterns in imaging. Furthermore, the presentation of CTD-ILD can vary based on patient-specific factors, such as age and what type of autoimmune response the body generates. (See, e.g., Autoimmune-Featured Interstitial Lung Disease, Vij, Rekha et al., CHEST, Volume 140, Issue 5, 1292-1299).

For IPF, there usually is no identifiable underlying disease. Thus, it is difficult or impossible for clinicians to assess whether a patient's presentation of solely LD symptoms alone means that the patient has IPF or that the underlying disease of a CTD-ILD disorder simply isn't being detected or not yet causing symptoms. Thus, some common approaches to diagnosis may involve radiologic imaging, as well as biopsy/histopathology. However, for IPF, serologic testing is typically inconclusive (while biopsy is often inconclusive for CTD-ILD).

Thus, based on current practices, clinicians' attempts to properly diagnose whether a patient has IPF or CTD-ILD is unusually difficult, and this can be especially problematic for older patients who may develop numerous disorders as they age that can complicate the process. As such, many patients wind up with a considerable number of clinic visits for different specialties, chest scans, blood tests, biopsies, etc. that are burdensome but still may not provide a clear diagnosis.

And, importantly, having a clear diagnosis as between IPF or CTD-ILD is not simply a matter of abstract classification—a patient's course of treatment can differ considerably as between the two, as well as their prognosis and symptom progression expectations. For example, if IPF is untreated or not treated correctly, it can progress rapidly, whereas CTD-ILD may exhibit a more variable progression. Misdiagnosis as between these two conditions can lead to incorrect or unnecessary treatments, progression of a disease, and unwarranted side effects. For example, the standard therapeutics prescribed for patients with CTD-ILD may include steroids, immunosuppressants, and similar medications that can actually worsen IPF.

Thus, there exists a need in the field to provide a more concrete and accurate way to differentiate between possible diagnoses that present similar symptoms and test/imaging results.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any of all aspects of the disclosure. Its sole purpose is to present some concepts of one or more aspects of the disclosure in a simplified form as a prelude to the more detailed description that is presented later.

In some aspects, the present disclosure can provide a method for distinguishing CTD-ILD from IPF. A preliminary diagnosis of a lung disease, a first data set corresponding to protein counts found in a blood sample, and a second data set corresponding to additional data from a patient may be obtained. The first data set and the second data set may be provided to a trained machine learning model and a predicted diagnosis of the lung disease may be determined. A recommended treatment may be outputted using the predicted diagnosis. A confirmation of the predicted diagnosis and the recommended treatment may be obtained.

In further aspects, the present disclosure can provide a system for classifying among similar disease. The system may include an electronic processor and a non-transitory computer-readable medium storing machine-executable instructions. When the instructions are executed by the electronic processor, they may cause the electronic processor to receive a user input indicating a preliminary diagnosis from a clinician of a set of possible disease for a given patient. A data set corresponding to data of the given patient may be obtained and the data set may be provided to a trained machine learning model. A predicted diagnosis may be determined from the set of possible diseases and a recommended treatment may be outputted using the predicted diagnosis. A confirmation of the predicted diagnosis and the recommended treatment may be obtained.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a flowchart illustrating an example interstitial lung disease classification process for a machine learning model.

FIG. 2 is a flowchart illustrating a process for updating a machine learning model.

FIG. 3 is a block diagram conceptually illustrating a system for the classification process using a machine learning model.

FIG. 4 is a flowchart illustrating a process for generating a trained differential diagnosis model.

FIGS. 5A and 5B are charts showing dataset filtering according to the inventors' validation studies.

FIG. 6 is a graph depicting ranking of proteins according to the inventors' validation studies.

FIG. 7 is a set of probability plots for demographic and test features according to the inventors' validation studies.

FIG. 8 is a set of probability plots for demographic and test features according to the inventors' validation studies.

FIG. 9 is a plot of variable importance according to the inventors' validation studies.

FIGS. 10A and 10B are plots of principal components analysis results according to the inventors' validation studies.

FIGS. 11A-11D are a set of plots of variation by training data source, according to the inventors' validation studies.

FIGS. 12A-12B are a correlated set of charts of results obtained from various differential diagnosis models according to the inventors' validation studies.

FIG. 13 is a sampling of patient/sample level results from the inventors' validation studies.

FIGS. 14A-14D is a set of graphs showing decision curve analyses comparing certain differential diagnosis models according to the inventors' validation studies.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

The disclosure in this detailed description section will include discussion of frameworks and associated general concepts that may be applicable to some or all of the more specific implementations contemplated herein; a discussion of the inventors' experiments and examples/prototypes used for validation; and descriptions of various embodiments or ways of implementing the systems and methods described herein. Thus, the descriptions of specific embodiments/implementations/examples should be understood to be capable of incorporating the more general frameworks and concepts as well as features of other specific embodiments, and vice versa.

At a general level, an advantage of the systems and methods of the present disclosure is the capability to provide objective, reliable, evidence-based, and clear aid in healthcare providers' efforts to differentiate IPF-type disorders and CTD-ILD-type disorders for specific patients. As noted above, while there may be symptom trends or test-result likelihoods that can be derived from larger scale comparisons between IPF-type disorders and CTD-ILD-type disorders, those trends and likelihoods do not hold up well when evaluating any specific patient in a real world clinical setting (that given patient may not present all diagnostically-pertinent symptoms, tests may be inconclusive, etc.). Furthermore, clinicians may not approach differential diagnosis in a way that elucidates pertinent information in the most effective sequence of testing and analysis (e.g., clinicians may initially avoid CT scans or biopsies if they suspect a different disease).

Thus, the present disclosure also contemplates taking the general improvements, algorithms, and advantages described herein and deploying them into practical implementations and systems, so as to leverage the improvements and algorithms for specific applications and real-world situations. For example, various example systems will be described below that apply the inventors' findings into networked systems that can aid several constituents of the healthcare system, including patients, clinicians, labs, radiology clinics, hospitals, electronic medical record and healthcare IT providers, payers and insurers.

### Example Classification Process for Machine Learning Model

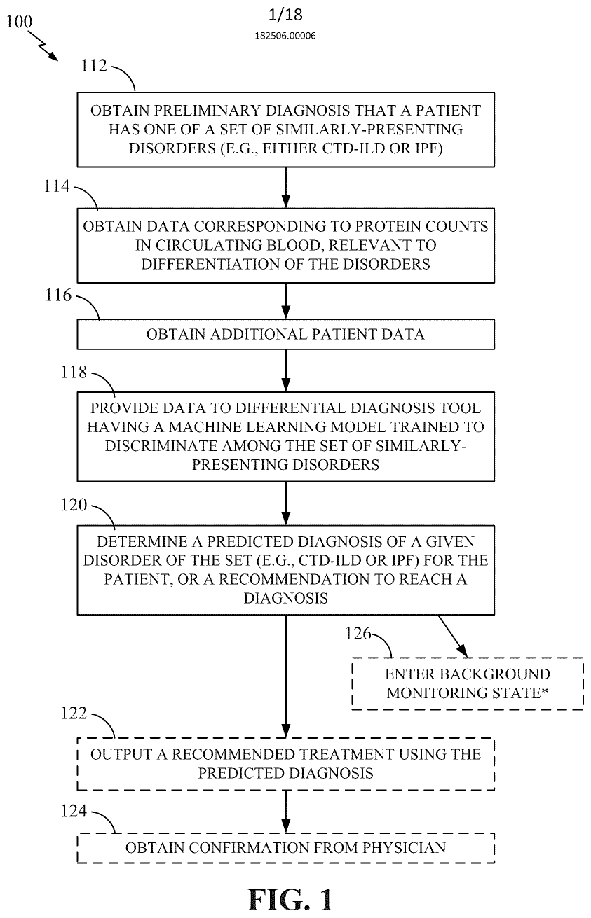

FIG. 1 is a flow diagram illustrating an example process 100 for classifying an interstitial lung disease using a machine learning model. In some embodiments, the process 100 can be utilized to differentiate between two or more possible diagnoses that fit the patient's symptoms and imaging/test results. In other embodiments, the process 100 can be thought of as classifying a given patient's disease state, from among a set of possibilities. As described below, a particular implementation can omit some or all features/steps, may be implemented in some embodiments in a different order, and may not require some illustrated features to implement all embodiments. In some examples, an apparatus can be used to perform the example process 100. However, it should be appreciated that any suitable apparatus or means for carrying out the operations or features described below may perform the process 100.

At step 112, the process 100 can obtain a preliminary diagnosis of a patient having one or more potential diseases that have been diagnosed as ILD-related, could potentially be ILD, or simply that the patient has symptoms similar to ILD-type symptoms. For example, the preliminary diagnosis may be ‘the patient likely has either CTD-ILD or IPF’ or ‘the patient presents ILD-type symptoms’ or merely an indication of the symptoms themselves and doctors' notes (which, could, for example be processed using a large language model (LLM) or other machine learning to derive a potential of ILD-relevant diagnoses or ILD-related symptoms). In some examples, a physician may input this preliminary diagnosis, it may be obtained from an electronic medical record, or it may be obtained from another user or source. In other examples, this preliminary diagnosis (e.g., a diagnosis of two or more possible disease states) may be obtained from another process that interprets results of a test or imaging, in a cascading approach utilizing more than one machine learning algorithm. In yet further examples, a patient may input information into a virtual aid, assistant, or advocate that postulates, suggests, or queries these types of symptoms or diagnoses.

At step 114, the process 100 can obtain data corresponding to protein counts found in the blood of the patient. In some embodiments, relative concentrations of each protein may be used. In other embodiments, absolute values for each protein count may be used. In some examples, the protein counts may be indicative of plasma protein biomarkers of plasma that traverses the patient's lungs. In some examples, a blood sample may be collected from a lab, clinic, etc. and tested for such biomarkers by known protein test methods, and the resulting data can be obtained. In other examples, a database such as a patient's electronic medical record may already contain the protein count data. To increase probability that the plasma has traversed the patient's lungs and/or to increase probability of biomarker detection, process 100 may suggest guidelines or protocols for sample collection, including for example any dietary, exercise/stress regimen, breathing exercises, rest, time of day, etc., and may generate the order for sample collection to be entered into a patient's EMR. References herein to “protein counts” may be understood as also contemplating other detection of proteins and/or other biomarkers in patients' circulating blood/plasma, such as when other ILD-related or non-ILD related sets of similarly-presenting diseases are being analyzed for differential diagnosis.

In further implementations of the process 100, step 114 may involve the performance, ordering, or direction of one or more of several types of tests for obtaining protein count information from patient blood samples. These tests may be optimized for differential diagnosis of classes of ILD disorders such as IPF vs. CTD-ILD, or existing tests may be utilized which can obtain large amounts of protein count information. In some examples, lateral-flow assays may be utilized for rapid, point of care diagnostic information (such as in a clinic visit, or when a healthcare organization or payer requires additional information before a clinician can prescribe a course of treatment for either IPF or CTD-ILD diagnosis), such as to detect biomarkers like IL-15 or MMP12 (or another biomarker or subset of biomarkers which, as described below, may have a high predictive ability to differentiate IPF from CTD-ILD), which may be part of the proteomic classifier described herein. Thus, these tests may provide simple, low-cost, rapid, out-patient verification of diagnoses for situations in which clinicians believe that they have made a confident diagnosis of a class of ILD disorder. In other circumstances, the LFAs may be utilized to gate (or supplement) further, more expensive or invasive testing (e.g., CT scans or biopsies). Other tests that may be utilized include those that would be performed in a more sophisticated or centralized laboratory, such as enzyme-linked immunosorbent assay (ELISA); mass spectrometry, multiplex immunoassays, Olink® proteomics panels, flow cytometry tests, etc. For example, when a patient presents with lung patterns via CT scan that could represent both IPF and CTD-ILD (or the CT scan is otherwise not conclusive of the diagnosis), a mass spec or ELISA test could be ordered. Regardless of test type(s), data may be standardized and/or normalized and integrated into a system operating process 100.

Thus, the present disclosure also contemplates, as practical implementations of the concepts presented herein, optimized tests for identifying specific biomarkers/protein counts for differentiation of ILD-like disorders. In some cases, the tests may allow for detection of multiple biomarkers at once (the biomarkers being selected from the examples, as further described below), detection of biomarkers other than antibodies, and better differential diagnosis as compared to customary serologic testing used to diagnose one or the other ILD-like disorders. Thus, the tests contemplated herein are more amenable to high-throughput lab tests as well as simple point of care tests, and thereby provide scalability and flexibility.

Furthermore, the tests contemplated herein would directly support an objective, reliable differential diagnosis of ILD-like disorders, whereas the types of serologic testing used to diagnose CTD-ILD disorders focus on autoantibodies or specific markers associated with autoimmune diseases. In other words, those serologic tests actually aim to diagnose the related autoimmune disease that may be associated with CTD-ILD, but not the CTD-ILD itself (versus other ILD-like disorders). Other prior tests may look for biomarkers for fibrosis, but these would not be disease-specific or differentiate among ILD types. And, these prior tests typically required correlation with clinical, imaging, and histological findings in a multi-disciplinary discussion. In contrast, the tests contemplated herein could allow for a single discipline (or fewer disciplines) to be involved in pinpointing a diagnosis of ILD type. Thus, healthcare organizations, clinics, and payers can more efficiently, confidently, and rapidly reach a point of confidence in determining the right therapeutic approach for a given patient, by utilizing initial diagnostic tools (patient assessment, and perhaps a CT or other scan) through a single clinic/clinician to reach a point of at least having identified ILD-related disorders as the general diagnosis, then can utilize the tests contemplated herein to avoid further testing and/or multi-disciplinary discussion in coming to a final, specific diagnosis. Additionally, in situations where members of a care team disagree on the diagnosis/treatment approach due to differences in opinion as to whether a patient has IPF or CT-ILD, testing contemplated herein can serve as an objective, evidence-based ‘tie breaker.’

At step 116, the process 100 can obtain additional patient data. In some examples, the additional patient data may include the patient's sex, race, and/or age. Moreover, the additional patient data can also include a patient's symptoms and other test results (e.g., blood pressure, relevant medical history, environmental risk factors, etc.), as reported by a physician and/or patient.

At step 118, the process 100 can provide data to a trained machine learning model. In some examples, both the data corresponding to protein counts obtained in step 114 and the additional patient data obtained in step 116 are provided to the trained machine learning model. The machine learning model may include a Support Vector Machine, a LASSO regression, various gradient-boosting algorithms, deep learning networks, a Random Forest (RF), and/or an imbalanced-RF, or may include ensemble approaches. The machine learning model(s) may have been trained in a fashion that accounts for uneven representation of these diseases in patient populations, as well as patient characteristics/demographics that may influence the presence, absence, or degree of any given biomarker, and the high dimensionality of the training data. In some examples, two, three, four, five, etc. models may be used in combination, or a user may choose one or more models to include in the machine learning model.

In some examples, multiple machine learning models may be available to process 100. For example, if a physician has ruled out one of three possible disease states, then the physician or other user can input data indicating that only two possible disease states are to be considered by the process. In this case, a machine learning model having two output channels will be selected, corresponding to the two possible disease states. In other embodiments, a physician may input a request to have both the two-disease-state model and the three-disease-state model utilized to further confirm the preliminary diagnosis. In some embodiments, the protein data may be standardized for multiple machine learning models, but the multiple models may have been trained utilizing various combinations of additional patient data. For example, while age, sex, and race may be available information in most cases, other risk factors may not be available information and/or uncertain. Thus, the process 100 can be configured to select one or more trained models that best correspond to available data and/or can discount the probative weight of uncertain factors. The machine learning models may have relatively equivalent performance metrics, including generalizability and discriminative signal strength.

Examples of training machine learning models can be found in the Examples section, below. However, as a general matter, the machine learning models may be trained utilizing training data that comprises: confirmed diagnosis (e.g., CTD-ILD versus IPF), preliminary diagnosis, as well as the categories of data provided in steps 114 and 116. Notably, machine learning models need not be trained utilizing ‘control’ data of patients that do not have CTD-ILD or IPF, as the machine learning models do not need to have an output channel of “no disease.” Thus, these machine learning models differ from more typical models for predicting a given disease state (typical disease prediction models are configured to answer the question: ‘does the patient have disease X’). In other words, certain embodiments of machine learning models of the present disclosure do not classify the presence or non-presence of a given disease, but rather are tailored to situations in which a physician has already preliminarily determined the patient has a disease (such as an ILD-related disorder) via patient examination and utilizing their analysis of patient symptoms, but is looking to differentiate which of a finite possible set of diseases it is.

At step 120, the process 100 can determine a predicted diagnosis of one of the possible disease states, such as a confirmation of whether the patient has CTD-ILD or IPF. At step 122, the process 100 can optionally output a recommended treatment using the verified diagnosis. For example, if the predicted diagnosis provided at step 120 indicated CTD-ILD, the recommended treatment provided at step 122 may involve immunosuppressive regimens. In some examples, the recommended treatment may be outputted via a user device, saved to a database, or sent to a patient or physician via a software system. At step 124, the process 100 can optionally obtain a confirmation from a physician. In some examples, a physician may place an order for a specific treatment upon confirmation of the verified diagnosis. The specific treatment may correspond to the recommended treatment provided in step 122. In other examples, step 124 may include the physician reviewing the verified diagnosis and either agreeing or disagreeing with the verified diagnosis and recommended treatment from steps 120 and 122, respectively.

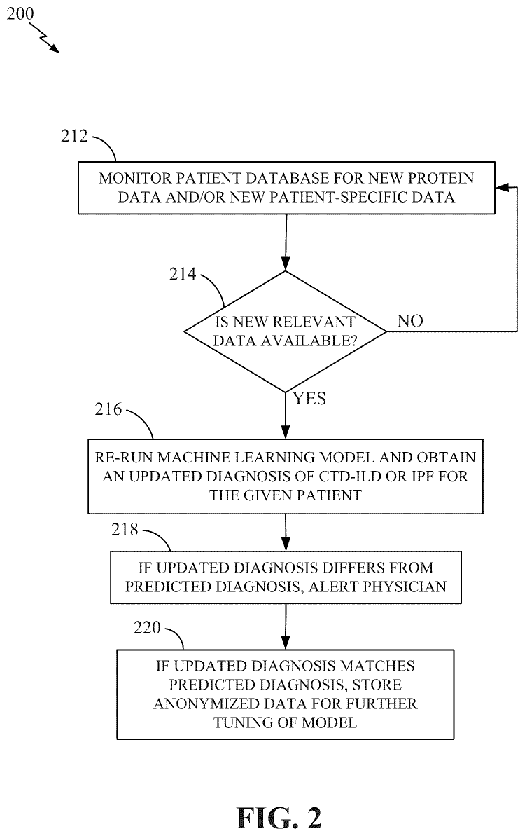

At step 126, the process 100 can optionally enter a background monitoring state. In some examples, the process 200 in FIG. 2 can be used in step 122 of process 100. FIG. 2 illustrates a flow diagram of an example process 200 for monitoring and updating a machine learning model. At step 212, the process 200 monitors a patient database for new protein data and/or new patient specific data. In some examples, this may include any data added to the system, such as updated symptoms and signs the patient may be experiencing, as well as updated protein data counts. In other examples, the new data may include the sex, race, and age of the patient, if not previously provided in step 116 of process 100. At step 214, the process 200 determines if there is any new relevant data available. If no relevant data is available, the process 200 returns to step 212. If there is relevant data available, the process 200 continues to step 216, where the machine learning model is re-run to now including the new protein data and/or new patient-specific data. Additionally, an updated diagnosis of CTD-ILD or IPF for the patient may be obtained.

At step 218, the process 200 determines if the updated diagnosis differs from the predicted diagnosis and alerts the physician. For example, the updated diagnosis determined at step 216 may be different from the predicted diagnosis determined at step 120 of process 100. The physician may be altered via a notification generated and sent to a device. At step 220, the process 200 determines if the updated diagnosis matches the predicted diagnosis and stores the anonymized data for further tuning of the machine learning model. For example, the updated diagnosis determined at 216 may be the same as the predicted diagnosis determined at step 120 of process 100.

### Example Systems, Networks, and Platforms

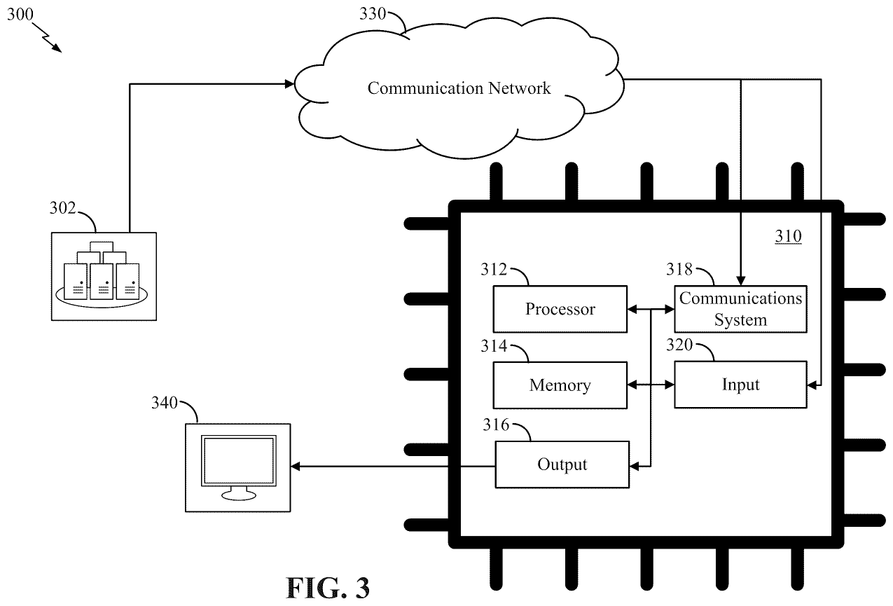

FIG. 3 shows a block diagram illustrating a system 300 for implementing the improvements, algorithms, and processes described herein, using one or more machine learning models according to some embodiments. In one respect, the system 300 can be thought of as a system that is configured to monitor and/or verify a physician's predicted diagnosis for a given patient. In another respect, the system 300 can be thought of as a system that guides and gates approaches to, first, diagnosing one of a subset of very similarly-presenting disorders, and second, prescribing treatment approaches for a diagnosed disorder from that subset. In other aspects, the process may provide a recommended treatment based on the classification between CTD-ILD or IPF.

The illustrated system 300 can, thus, include components that are patient-facing (e.g., patient portals) or patient-specific (e.g., a patient's EMR); components that are clinician facing (e.g., workstations and clinician interfaces that provide aid in differential diagnoses); and components that have a more ‘background’-focused role, such as drawing data from multiple sources, monitoring for new data, issuing prescription/test orders to outside networks (e.g., pharmacy networks, radiology clinics, etc.), and computing classification results.

As shown, the computing device 310 can be a device, network, or other resource that includes an integrated circuit (IC) or processor for computation, such as a server, cloud resource, or any suitable computing resource. In some examples, the computing device 310 can be a special purpose device (e.g., a machine or co-processor, or including an ASIC) that can efficiently compute differential diagnoses by running a machine learning model, but within an environment that allows for security, privacy, and compliance with healthcare-related regulation (such as HIPAA, anti-kickback rules, payer interventions, etc.). Thus, the processes 100 and 200 described in FIGS. 1 and 2 can be implemented for or by a special purpose device.

In the system 300, a computing device 310 includes a data communications link such that it can obtain or receive a dataset. The dataset can be a set of protein counts found in the blood 302, or any other suitable dataset for running processes such as process 100. For example, the dataset can include data obtained from a laboratory or a preexisting dataset. Also, in some examples, the dataset can include a training dataset to be used to classify lung diseases for a machine learning model. In some examples, the dataset can be directly applied to a machine learning model. In other examples, one or more features can be extracted from the dataset and then only the relevant features can be applied to the machine learning model. The computing device 310 can receive the dataset, which is stored in a database, via communication network 330 and a communications system 318 or an input 320 of the computing device 310.

The computing device 310 can include a memory 314. The memory 314 can include any suitable storage device or devices that can be used to store suitable data (e.g., the dataset, a trained machine learning model, a neural network model, a software application running a user interface, an integration to an electronic medical record, etc.) and software instructions that can be used, for example, by the processor 312. The memory 314 can include a non-transitory computer-readable medium including any suitable volatile memory, non-volatile memory, storage, or any suitable combination thereof. For example, memory 314 can include random access memory (RAM), read-only memory (ROM), electronically-erasable programmable read-only memory (EEPROM), one or more flash drives, one or more hard disks, one or more solid state drives, one or more optical drives, etc., or may simply be an apportioned cloud, network, or other resource. In some embodiments, the processor 312 can execute at least a portion of processes 100 and 200 described above in connection with FIGS. 1 and 2

The computing device 310 can further include a communications system 318. The communications system 318 can include any suitable hardware, firmware, and/or software for communicating information over the communication network 330 and/or any other suitable communication networks. For example, the communications system 318 can include one or more transceivers, one or more communication chips and/or chip sets, etc. In a more particular example, the communications system 318 can include hardware, firmware and/or software that can be used to establish a Wi-Fi connection, a Bluetooth connection, a cellular connection, an Ethernet connection, etc.

The computing device 310 can receive or transmit information (e.g., dataset 302, a diagnosis output 340, a trained neural network, etc.) and/or any other suitable system over a communication network 330. In some examples, the communication network 330 can be any suitable communication network or combination of communication networks. For example, the communication network 330 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 330 can be a local area network, a wide area network, a public network (e.g., the Internet), a private or semi-private network (e.g., a corporate or university intranet), any other suitable type of network, or any suitable combination of networks. Communications links shown in FIG. 3 can each be any suitable communications link or combination of communications links, such as wired links, fiber optic links, Wi-Fi links, Bluetooth links, cellular links, etc.

In some examples, the computing device 310 can further transmit an output connection 316 to a user interface 340. The output 316 connection may be part of or rely upon a network connection such as the communication link 330, but alternatively may be a separate connection such as, e.g., a private connection to a healthcare organization's electronic medical record system or may include other connections such as an email server. The form of output connection 316 may depend upon the form of data to be provided to a user as well as where the computing device 310 resides. For example, if the computing device 310 is hosted by the laboratory that runs the blood test to generate the protein data, then the output 316 could simply be an indication of likelihood of which of the possible disease states corresponds to the blood sample that was tested. As another example, if the computing device 310 is hosted by a healthcare organization or clinic, the output may comprise all or a portion of a user interface directed to the treating physician. In some embodiments, the output connection 316 can transmit a diagnosis of either CTD-ILD or IPF, a recommended treatment, a user alert, and/or other information. In other examples, the output 316 can include a display to output a prediction indication. In some embodiments, the display 316 can include any suitable display devices, such as a computer monitor, a touchscreen, a television, an infotainment screen, etc. to display the report, the diagnosis output 340, or any suitable result of a diagnosis output 340. In further examples, the diagnosis output 340 or any other suitable indication can be transmitted to another system or device over the communication network 330.

In further examples, the computing device 310 can include an input connection 320. The input connection 320 can be coupled to a communication link such as network 330 for receipt of data from remote locations (e.g., protein count data, etc.) or may be an integration to a locally-controlled electronic medical record or other healthcare software. For example, the input connection 320 may receive a set of protein counts corresponding to the dataset 302. In other examples, the input 320 can include any suitable input devices (e.g., a keyboard, a mouse, a touchscreen, a microphone, etc.) and/or the one or more sensors that can produce the raw sensor data or the dataset 302.

In the Examples section, below, further examples are provided that describe various methods of training machine learning models to differentiate among possible disease states indicated by a physician. The specific examples are not limiting of the scope of this disclosure, but rather illustrate several general principles that guide the creation of machine learning models for use in process 100 and/or process 200, via systems such as system 300.

For example, in some embodiments a dataset may be obtained that provides a wide-ranging set of information relating to patients that were given confirmed diagnoses of one of a set of similar diseases. This initial training data set may include test results of a proteomics analysis of the patients' blood samples, but may also include information such as patient age, patient sex, patient race, and other information such as recorded vitals (e.g., average heart rate, blood oxygen levels, blood pressure, lung volume, etc.) and/or other relevant risk factors. Furthermore, the training data set may include a physician's preliminary diagnosis, if different from the final confirmed diagnosis.

Optionally, the dataset may be preprocessed to extract relevant features and or sparsify the data. For example, where it is well known that certain protein markers are highly correlative to all of the disease states of interest, they can be removed from the dataset. Similarly, where none of the disease states are meaningfully correlated with certain data elements (e.g., environmental risk factors are not relevant), or a model is desired that can operate solely on confirmable laboratory information, associated fields of the data set can be removed.

Next, a machine learning model may be configured to have input channels corresponding to the data fields of the dataset, and output channels that are limited to the set of similar disease states from which the model will be trained to differentiate. For example, the model may be programmed to have input channels corresponding to the protein data (whether alone or in combination with the additional data), and output channels that correspond only to the set of disease states of interest (e.g., embodiments may exclude a ‘no-disease’ output channel). Then, the model may be trained on the dataset.

The result of training the model will depend to some extent on the type of model utilized. In some embodiments, training the model can result in not only a trained model, but also a listing of the discriminatory power of each field of the training dataset relative to the decision of which disease set of the finite set of disease states is most likely. Notably, the inventors have found that the biomarkers that best discriminate between disease states is very often not the same as or similar to the biomarkers that would traditionally be used in a simple, binary diagnostic of one particular disease.

In a refinement step, fields of the dataset that have least discriminatory power can be pruned, and the model re-run and validated to assess impact on accuracy. This process can be continued sequentially until a threshold number of proteins is reached or a threshold accuracy is reached. In some embodiments, the threshold number of proteins may be pre-set by a user or may correlate to a desired test. For example, if a given classification process is desired that can utilize information from a more simple test (e.g., a lateral-flow test strip, blot test, or lab-developed test) or from more cost-effective reagents, the threshold number of proteins may be limited by the capabilities of such tests. In association with the thresholding step, a further refinement may include removal of proteins that cannot readily be tested in a given environment or with available resources, and the pruned proteins iteratively added back to the model until a desired accuracy is reached. For example, as described in the attached appendices, the inventors found that protein counts used for diagnostic purposes could be limited to 50 or fewer specific proteins, such as 37 proteins, or even fewer—depending in some cases on what additional data is used in conjunction with protein data to train the model.

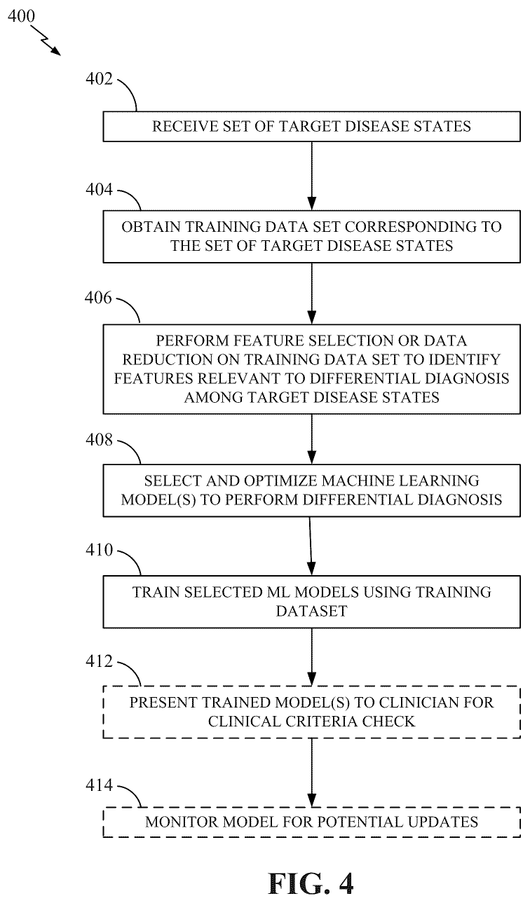

Referring now to FIG. 4, an example process 400 is shown for generating and optimizing a trained model for differentiating among disease states that have similar clinical presentations. In some embodiments, this process 400 may be performed to generate a trained model for a given disease differentiation for a given population, and then deployed for use in a process such as described with respect to FIGS. 1 and 2. In other words, process 400 may be a method of making a system or algorithm to be deployed in the embodiments contemplated herein. In other embodiments, process 400 may be a more dynamic method that can generate or refine trained models on the fly for particularized diagnostic situations by a healthcare system or provider.

At step 402, a set or subset of disease states may be identified. (In the Examples section, CTD-ILD and IPF were selected, but further refinement into subtypes of ILD-related disorders is contemplated, as well as non-ILD related disorders which may present similar diagnostic difficulties as ILD-related disorders). A user may input the set or subset of disease states by specifying the possible outputs of the trained model (e.g., the target subset of disease states will be: “Disorder 1,” “Disorder 2,” or “Disorder 3”), or process 400 may derive the possible disease states by applying natural language processing to information such as doctor's notes in an EMR, a transcription of a patient visit, etc. In yet further embodiments, process 400 may utilize a large language model or similar network to periodically review scientific literature publications to identify disease states that have similar presentation of symptoms (but different treatment) and for which researchers and clinicians seem to have difficulty differentiating. As such disease states are identified, they can be provided as suggestions or prompts to an operator of process 400.

At step 404, data may be collected to serve as a training dataset. In some embodiments, the data should include information labeling each record as being associated with one of the target disease states; each record may also be normalized and standardized, and/or pruned to eliminate irrelevant or extraneous/non-common data. For example, the data records that form the training dataset may include anonymized patient health data records for patients who were confirmed to have one of the target disease states. The data records may include fields that reflect information on: final diagnosis; radiology images (e.g., CT, MRI, or x-ray); serologic tests performed, and results; blood tests and results; biopsies performed and results; measures of symptomatic presentation such as pulmonary function tests, exercise tests, or cardiopulmonary tests; bronchoscopy tests, such as biopsies or fluid collection; pathology and histology analyses; general patient demographics (such as sex, age, cardiopulmonary risk factors, health history, geography, etc.). Test result data may include biomarker data, such as -omics test results or specific antibody/protein assays or panels. In some embodiments, treatment approaches may also be included in such data records, along with outcome information.

Where data records are amalgamated from multiple sources, or were generated using different records techniques (e.g., different EMR types or formats, clinical trial data records, etc.) they may require modification to conform field identifiers, data formats (e.g., decimal places for test results; CT image file type, cropping, etc.), etc. or may benefit from value adjustments such as up/down sampling of resolution and binning of test results to account for variation in test sensitivity. In further embodiments, where not all records have data for any given field, process 400 may eliminate stray fields in order to promote homogenous content of the data set, impute values based on similarity to other records, or adjust weighting of the model to account for missing and non-homogenous data. In further embodiments, process 400 may cull the available data to create an appropriate proportion of data records as between the target disease states to reflect their relative prevalence among demographic populations.

At step 406, process 400 may optionally perform certain exploratory analyses to determine whether feature selection or data dimensionality reduction would be appropriate. For example, some or all data records associated with each disease state may be analyzed to remove features that may be diagnostically relevant to the disease states from a de novo standpoint, but which may not be diagnostically relevant to a differential diagnosis as between the subset of target disease states. Thus, counter-intuitively, process 400 may actually remove data points from the training dataset that would be strong predictors of the disease states, if they are strong predictors of all or multiple of the subset of target disease states.

To select features, reduce data dimensionality, and/or emphasize higher-order and non-linear relationships, a number of algorithms may be utilized. As noted above, however, the present disclosure contemplates both general use of these algorithms as well as tailoring of these algorithms to the specific target disease state subsets and goals of process 400. For example, an initial step of eliminating features that are identical across all data records may be applied. Alternatively or additionally, a recursive feature elimination process may be employed, but instead of a customary process in which features are maintained or culled based on presence or absence of a given disease state, the feature elimination is forced to account for only disease states (and not the absence of any given disease state). Thus, a model may be iteratively trained and features with lowest discriminatory power (which may not be the same as general classification/identification power) may be removed until a point is reached at which the least-discriminatory features remaining are still above a given threshold. In other examples, rather than eliminating features, they may be preserved (e.g., in case their correlative relationship to other data fields may still be important) but given reduced weighting. For example, a regularized regression method such as Elastic Net, which combines L1 and L2 penalties of the LASSO and ridge methods can be employed as an alternative to pure feature elimination. In the case of differentiation of similarly-presenting disease states, it may be particularly helpful to preserve a given biomarker even if it is common among all of the target disease state (e.g., it is related to a shared biological pathway) but its presence in conjunction with other markers could still be very discriminatory (such as in situations where the different target disease states relate to overlapping pathways).

Several modifications, changes, and adaptations of RFE algorithms may be employed, which can cause them to perform in ways that are more clinically and biologically appropriate to the tasks and goals described herein. For example, customized weighting of the RFE may be performed, which can be tailored to assign higher or different weights to features associated with comparatively less prevalent target disease states, so as to avoid overfitting to the majority target disease state(s). This approach may make it more likely that features important for discriminating less-represented conditions or demographics are not prematurely eliminated. As another example, cross-validation approaches may be employed to examine how the feature elimination is affecting the model relative to certain populations represented in the dataset, features that are likely not to be directly relevant (e.g., insurance status), and/or features that are known to reflect clinically-determinative presentations. This may be beneficial in circumstances in which multi-cohort data records are used, data records are obtained from multiple sources (e.g., which may reflect inherent biases of local clinicians and institutional approaches, or impact of socio-economic factors like insurance coverage on testing and treatment) or multiple demographics. As another example, RFE may be combined with or embedded into a gradient-boosting process or other ensemble method, so that features are ranked according to overall loss/gain in the ensemble performance, in order to leverage the strengths of the overall ensemble learning. As noted above, given that the presentation and symptoms of certain similar disease states (like sub-classes of ILD-related

In other examples, these feature selection/optimization processes may be performed on individual subsets or overlapping subsets of the data in each record, to account for relationships between and among data types. For example, RFE could be performed solely on -omics data such as protein counts, but could also be performed on -omics data in combination with demographic data and features extracted from, or labels added to, imaging results, etc. Or, feature reduction could be performed on test result data, but cross validated against models trained on all or more fields of the training dataset. Thus, given the heterogeneity of data points and the known variation in how similar target disease states present, as well as circumstances in which datasets for less prevalent disease sets may be small, it can be important to examine which features are “stable” in the sense of ensuring that they remain discriminatory across training dataset sampling (to ensure the features are not simply a result of overfitting).

At step 408, model types, combinations, and ensembles can be selected and optimized. For example, during the process of feature selection at step 406, individual model types may be utilized and retrained during elimination or down-weighting of less relevant features, such as Random Forest models, Support Vector Machines, Gradient Boosting, ensembles, etc. The actions taken at step 406 may entail some basic initial hyperparameter setting. At step 408, however, more comprehensive model initialization and hyperparameter tuning may be performed to optimize the model's performance specifically for final diagnostic differentiation applications. For example, once features have been selected or modified by weighting, hyperparameters can be modified to tune each model (whether to be used alone or as part of an ensemble), such as: setting the number of trees, tree depth, class weights, etc. for a RF model; determining kernel type, regularization, or gamma values for SVM models; etc. Thus, samplings of the training dataset can be pulled to be used to measure model performance/accuracy as various hyperparameters are changed. Additionally, the models can be compared to one another, and compared to various combinations/ensembles of models, to determine which may be most useful for differentiation among target disease states.

At step 410, process 400 may also involve specific training and validation of the models. This may involve splitting the dataset into training and validation subsets, and training the models on the data using the selected features. In some embodiments, techniques as described above (e.g., weighted loss functions, balanced sampling, etc.) may be utilized to handle class imbalance or preserve importance of features that are known to be differential. This may also entail ensemble optimization, such as tuning ways to combine predictions from multiple models (e.g., voting, stacking, etc.) and integrate outputs.

At step 412, process 400 may optionally present one or more models, ensembles, or settings to a clinician or other expert so that thresholds can be adjusted to ensure they match clinical relevance.

At step 414, process 400 may then enter a state of monitoring performance of the finalized model/ensemble, such as described above with respect to FIG. 2.

### Examples and Validation Experiments

The inventors discovered through their research that differences in immune responses are present between those with CTD-ILD and IPF, such that a blood-based proteomics approach to establish a classifier would be able to correctly distinguish and molecularly characterize these two classes of ILD-related disorders. The following discussion will pertain to the inventors' research and validating experiments, but it should be understood that these results and the specific classifiers developed in these studies are not limiting of the types of processes and systems described above.

Initially, the inventors determined that a blood-based test would provide several advantages (e.g., versus tissue biopsies or lung fluid analysis). Circulating plasma is easily acquired, sampling blood that traverses the entire lung, and a proteomic approach simultaneously examines large numbers of proteins. Plasma protein biomarkers have previously been successfully associated with the de novo diagnosis of IPF, so proteomic blood testing would have some similarities to these findings. And, the inventors determined that plasma proteins are also attractive to identify CTDs because they can provide representative cell activities involved in autoimmunity. However, the inventors' experiments achieved the novel discovery of differential diagnosis as between types of ILDs that otherwise elude or confound diagnosis by existing tests.

From their research, the inventors determined that a combination of machine learning models applied to high-throughput proteomic data from circulating plasma could establish a classifier to differentiate patients with auto-immune driven CTD-ILD from IPF. The proteins involved could provide insights into pathobiological mechanisms. And, the classifier is able to make its assessment based on single-patient samples. This reflects the case-by-case clinical practice environment, overcomes the proprietary nature of single-center cohort collections, and surmounts the limitations of any single machine learning model.

The inventors' research drew from a variety of sources to generate a training dataset: the Pulmonary Fibrosis Foundation (PFF) Patient Registry, University of Virginia (UVA), and University of Chicago (UChicago) cohorts included both IPF and CTD-ILD patients. Additionally, the University of California at Davis (UC-Davis) and U.K. RECITAL clinical trial provided IPF and CTD-ILD patients, respectively.

Peripheral blood was collected in EDTA tubes (from patients all centers, except for RECITAL samples in which were collected in Heparin tubes. Plasma was isolated, aliquoted, and stored at −80° C. Frozen plasma from all centers was consolidated and randomized based on center, age, sex, and race at the time of plating and processed in a single batch to mitigate batch effects. The Olink® Explore 3072 panel (Uppsala, Sweden) was used to generate semi-quantitative proteomic data for 2939 analytes covering 2921 proteins. Proteins below the lower detection limit were imputed to the lowest observed value. Protein data were normalized to minimize both intra- and inter-assay variation. Protein levels are summarized to NPX (Normalized Protein eXpression) in Log 2 scale for data aggregation across plates.

Two hundred and forty samples were selected as the training cohort from the PFF registry, with equal representation of 60 male and 60 female patients from both CTD-ILD and IPF categories. This approach ensured both diagnosis and sex distribution neutrality. This process was repeated 100 times to ensure sufficient representation of sample heterogeneity across PFF cohort. The training cohorts formed through this subsampling strategy were then utilized for various analyses, including two-sample comparisons, protein feature selection, implementation of machine learning models for testing of independent cohorts and single-sample classification.

Detailed demographic and clinical characteristics of each cohort were also recorded and included in the training dataset, including the features shown in Table 1, below. Significant differences in characteristics included age, race, and higher proportion of males in the IPF group compared to CTD-ILD. CTD-ILD cases had significantly lower Gender-Age-Physiology (GAP) scores than IPF in both training and test cohorts. However, ROC analysis showed that GAP score only mildly distinguished between CTD-ILD and IPF in both training (AUC 0.71) and test (AUC 0.68) cohorts.

TABLE 1
Demographic and clinical characteristics of IPF patients in PFF Patient Registry:

| | IPF in PFF | IPF outliers | |
|---|---|---|---|
| | training | in PFF Patient | |
| | cohort | Registry | p-value |
| Sample size | 881 | 25 | NA |
| Sex (Male/Female ) | 668/213 | 19/6 | chisq p = 1 |
| Age (Mean/Median) | 70.9/70.2 | 71.0/71.0 | t-test p = .672 |
| Race (Black/Hispanic/Unknown/White | 10/22/18/831 | 1/1/0/23 | NA |
| % predicted FVC (Mean/Median) | 67.7/66.7 | 69.6/66.5 | t-test p = 0.492 |
| % predicted DLCO (Mean/Median) | 40.5/39.0 | 39.5/43.8 | t-test p = 0.471 |
| GAP Score (Mean/Median) | 4.5/4.0 | 4.4/4.5 | t-test p = 0.579 |
| GAP Stage (Low/Medium/High ) | 178/454/198 | 5/16/2003 | chisq p = 0.389 |
| Height (Mean/Median) | 67.9/68.0 | 67.3/67.0 | t-test p = 0.408 |
| Smoking History (Yes/No ) | 563/318 | 16/9 | chisq p = 1 |
| GERD (Yes/No/Unknown) | 553/315/13 | 14/10/1 | chisq p = 0.745 |
| Survival (alive/death/transplant) | 530/233/111 | 17/6/2 | Wald test p = 0.575 |

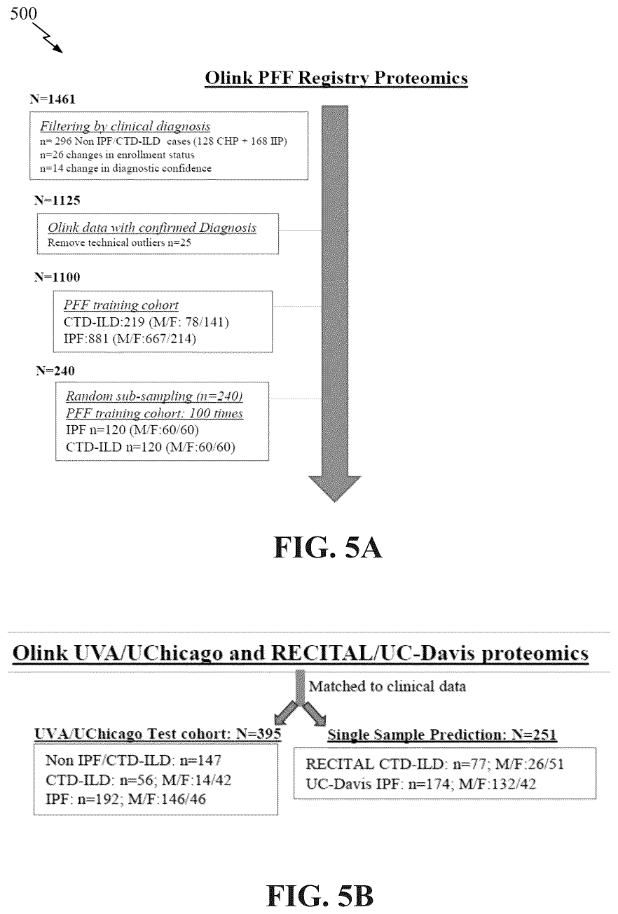

Olink® proteomic data were generated from PFF Registry (N=1461), UVA/UChicago testing (N=402), and RECITAL/UC-Davis (N=263) cohorts, as shown in FIG. 5. After applying exclusions filters, the proteomics datasets with matched clinical phenotype included: 881 IPF (M/F: 667/214) and 219 CTD-ILD (M/F: 78/141) cases for training from PFF (FIG. 1A); 192 IPF (M/F: 146/46) and 56 CTD-ILD (M/F: 14/42) for testing from UVA/UChicago; and 174 IPF (M/F: 132/42) from UC-Davis and 77 CTD-ILD (M/F: 26/51) cases from RECITAL study for single-sample classification. (PCA analysis identified 25 IPF cases in the PFF cohort as proteomic outliers. They showed no significant clinical or demographic differences compared to the rest of the PFF cohort, so these cases were removed for concerns over unknown technical variations.) Detailed subgroups constituting the bulk of PFF and UVA/UChicago CTD-ILD cases are listed in Table 2. These subgroups include systemic sclerosis, rheumatoid arthritis (RA), idiopathic inflammatory myositis and others.

TABLE 2
Sub-groups of CTD-ILD in PFF training and UVA/UChicago testing cohorts.

| | | | False Negative |
|---|---|---|---|
| | PFF (%) | UVA/UChicago (%) | case (%) |
| Ankylosing spondylitis | 1 (0.46) | 0 (0) | 0 (0) |
| Idiopathic inflammatory myositis | 46 (21) | 11 (19.64) | 9 (1) |
| Mixed connective tissue disease | 17 (7.76) | 10 (17.86) | 10 (1) |
| Rheumatoid arthritis | 50 (22.83) | 21 (37.50) | 28.5 (6) |
| Sjogren | 15 (6.85) | 7 (12.50) | 14.3 (1) |
| Systemic lupus erythematosus | 13 (5.94) | 3 (5.36) | 0 (0) |
| Systemic sclerosis/scleroderma | 70 (31.96) | 3 (5.36) | 0 (0) |
| Polymyalgia Rheumatica | 0 (0) | 1 (1.79) | 100 (1) |
| ANCA - vasculitis/pulmonary | 7 (3.2) | 0 (0) | 0 (0) |
| capilaritis/vasculitis | | | |

Two-group comparison using random subsampling from a balanced group of 240 cases with matched diagnosis and sex distribution, identified 88 proteins as significantly different between CTD-ILD and IPF in the training cohort (Table 3, FDR<0.05). GSEA pathway analysis showed that complement and coagulation cascades in IPF and nonspecific immune responses in CTD-ILD including interferon induction, host-pathogen interaction and pattern recognition pathways were increased respectively. Table E4 lists all 18 significant pathways of GSEA analysis with adjusted p-value<0.05.

TABLE 3

| | CTD-ILD | IPF | logFC | pval | p. adj |
|---|---|---|---|---|---|
| IL15 | 0.76366043 | 0.25042598 | −0.5132345 | 1.70E−06 | 0.00018078 |
| LGALS4 | 1.09104449 | 1.67899738 | 0.58795288 | 2.13E−06 | 0.00020341 |
| MMP10 | 0.95227509 | 1.55913299 | 0.6068579 | 2.40E−06 | 0.00022831 |
| POMC | −0.0853413 | 0.69558448 | 0.78092581 | 2.68E−06 | 0.00027403 |
| CRLF1 | 0.58906641 | 0.31881282 | −0.2702536 | 8.90E−06 | 0.00039685 |
| MMP12 | 0.83097582 | 1.48388451 | 0.65290869 | 5.14E−06 | 0.0005376 |
| SOST | 0.3457585 | 0.75012893 | 0.40437043 | 8.30E−06 | 0.0005578 |
| ADGRG1 | 1.08727516 | 2.09057767 | 1.00330251 | 2.31E−05 | 0.00100768 |
| KLRF1 | 0.27910053 | 0.7759553 | 0.49685477 | 2.90E−05 | 0.00139936 |
| SOD2 | 0.6946632 | 0.31035574 | −0.3843075 | 6.73E−05 | 0.00143575 |
| BPIFB1 | 0.77277515 | 1.30301757 | 0.53024242 | 4.22E−05 | 0.00163296 |
| CPM | 0.30369986 | 0.65152603 | 0.34782617 | 4.32E−05 | 0.00183642 |
| KRT19 | 2.27642263 | 2.91644425 | 0.64002163 | 5.49E−05 | 0.00205546 |
| WNT9A | 1.30775053 | 1.62529983 | 0.31754929 | 4.51E−05 | 0.00210048 |
| POF1B | 0.72489787 | 1.11411933 | 0.38922146 | 6.75E−05 | 0.00230851 |
| DDC | 0.21851876 | 0.67589591 | 0.45737715 | 5.80E−05 | 0.0025392 |
| CCDC80 | 1.02043841 | 1.41462281 | 0.3941844 | 0.00010398 | 0.00300873 |
| EDIL3 | −1.3079798 | −0.9342297 | 0.37375016 | 0.00021353 | 0.00497226 |
| TRIM21 | 1.27942503 | 2.33480909 | 1.05538406 | 0.0002752 | 0.00500759 |
| CCL27 | 0.56364043 | 0.94371248 | 0.38007206 | 0.00031696 | 0.00539296 |
| SCGB1A1 | 1.41887838 | 1.78556333 | 0.36668496 | 0.00030962 | 0.00577119 |
| ADAMTS16 | 0.91676658 | 1.22188051 | 0.30511393 | 0.00027774 | 0.00643141 |
| AGR2 | 1.96811868 | 2.63110012 | 0.66298144 | 0.00029599 | 0.00673581 |
| ITGB6 | 0.85110546 | 1.19855234 | 0.34744688 | 0.00027699 | 0.00722018 |
| GALNT5 | 0.82351764 | 1.118337 | 0.29481936 | 0.0003827 | 0.00751326 |
| MLN | 1.78742268 | 2.41678421 | 0.62936153 | 0.0004665 | 0.00775326 |
| ELN | 1.21798015 | 1.60200629 | 0.38402614 | 0.00035018 | 0.00844405 |
| CEACAM5 | 0.70470988 | 1.2753968 | 0.57068692 | 0.00047593 | 0.00854238 |
| SELPLG | −0.2073187 | 0.01702758 | 0.22434623 | 0.00062565 | 0.00891763 |
| FUT3_FUT5 | 0.28776861 | 0.69376013 | 0.40599153 | 0.00044913 | 0.00906363 |
| CDCP1 | 1.46593267 | 1.84616261 | 0.38022994 | 0.00045197 | 0.00970484 |
| STC2 | 1.01961453 | 0.78129079 | −0.2383237 | 0.00096866 | 0.00980061 |
| CD93 | 0.68102682 | 0.39809733 | −0.2829295 | 0.00104863 | 0.00991263 |
| VWC2 | 0.49090448 | 0.817595 | 0.32669052 | 0.00053609 | 0.01106821 |
| FCER2 | 0.16143498 | 0.66222943 | 0.50079444 | 0.00059574 | 0.01127667 |
| FNDC1 | −0.2129802 | 0.18005374 | 0.39303393 | 0.0016876 | 0.01198384 |
| KDR | 0.15409583 | −0.0381184 | −0.1922142 | 0.00119123 | 0.01208418 |
| AREG | 1.20022423 | 1.65169525 | 0.45147103 | 0.00061255 | 0.01237824 |
| AOC3 | 0.58709974 | 0.79956963 | 0.21246988 | 0.00064615 | 0.01366482 |
| IGFBPL1 | 0.66918888 | 0.95219827 | 0.28300938 | 0.00129772 | 0.01440465 |
| TNFRSF13B | 0.28628312 | 0.59337702 | 0.3070939 | 0.00116166 | 0.01540021 |
| CXCL17 | 1.89976488 | 2.27729073 | 0.37752586 | 0.00108505 | 0.0158176 |
| EPS8L2 | 0.94771307 | 1.21086273 | 0.26314966 | 0.00100224 | 0.01591324 |
| FCRL6 | 0.02907733 | 0.53606072 | 0.50698338 | 0.00111451 | 0.01598646 |
| NELL2 | 0.21561487 | 0.4533786 | 0.23776373 | 0.0012395 | 0.01861658 |
| CD22 | −0.0283124 | 0.45664784 | 0.48496023 | 0.00220688 | 0.0216852 |
| GIP | 0.12074648 | 0.5481745 | 0.42742803 | 0.00175689 | 0.02171141 |
| TAFA5 | 0.88557584 | 1.18927208 | 0.30369624 | 0.0018864 | 0.02184352 |
| CGB3_CGB5_CGB8 | 0.35653183 | 0.62843333 | 0.2719015 | 0.00177609 | 0.02303714 |
| PRSS8 | 1.08434215 | 1.33957512 | 0.25523297 | 0.00194131 | 0.02357381 |
| KLRB1 | 0.11992835 | 0.4111452 | 0.29121685 | 0.00205778 | 0.02532465 |
| IL10 | 1.45043278 | 0.86734697 | −0.5830858 | 0.00287972 | 0.02754635 |
| CXCL14 | 1.16665688 | 1.54733638 | 0.3806795 | 0.0025737 | 0.02767491 |
| IL17D | 0.98715093 | 1.23324676 | 0.24609583 | 0.00270244 | 0.02790667 |
| FCRL2 | 0.16852036 | 0.54122983 | 0.37270947 | 0.00267315 | 0.02806077 |
| CLEC4G | 0.72294435 | 0.52235233 | −0.200592 | 0.0034655 | 0.0281847 |
| CRTAC1 | 0.65083394 | 0.84459315 | 0.19375921 | 0.00227122 | 0.02869507 |
| DCN | 0.23261513 | 0.39944502 | 0.16682989 | 0.00242953 | 0.02904114 |
| ICAM5 | 1.4124999 | 1.7184745 | 0.3059746 | 0.00260089 | 0.02939991 |
| CBLN4 | 0.42121638 | 0.66301133 | 0.24179496 | 0.00266387 | 0.02946218 |
| LEFTY2 | 0.53481042 | 0.89876571 | 0.36395529 | 0.00318671 | 0.03198526 |
| TCL1A | 1.63463378 | 2.52821604 | 0.89358226 | 0.00344417 | 0.0324491 |
| APOD | 0.48679103 | 0.26474952 | −0.2220415 | 0.00583951 | 0.03348241 |
| TNFRSF11B | 0.70626134 | 0.9559863 | 0.24972496 | 0.00302753 | 0.0336501 |
| CNTN3 | 0.17403627 | 0.38703861 | 0.21300234 | 0.00504037 | 0.03549145 |
| LYPD3 | 0.19155051 | 0.45188543 | 0.26033492 | 0.00300443 | 0.035939 |
| OXCT1 | 2.31321413 | 1.97942042 | −0.3337937 | 0.00326636 | 0.03599443 |
| S100A14 | 0.29343433 | 0.58806032 | 0.29462599 | 0.00404617 | 0.03723494 |
| CRH | −0.6719602 | −0.1029767 | 0.56898351 | 0.0040265 | 0.03771305 |
| SERPINA9 | −0.2466113 | 0.22564979 | 0.47226112 | 0.00398282 | 0.03810815 |
| ROBO2 | 0.62254052 | 0.81070131 | 0.18816079 | 0.00445524 | 0.03844732 |
| EDA2R | 1.60661702 | 1.94594462 | 0.3393276 | 0.0040968 | 0.03906651 |
| GNLY | 0.15241913 | 0.54204498 | 0.38962585 | 0.00488933 | 0.03915586 |
| FCRL1 | −0.1391095 | 0.26274251 | 0.40185205 | 0.00504247 | 0.04028458 |
| PRND | 0.48159475 | 0.02433849 | −0.4572563 | 0.00670931 | 0.04063486 |
| NBL1 | 0.72403043 | 0.88835644 | 0.16432602 | 0.00515439 | 0.04064715 |
| CDH1 | 0.4054298 | 0.59303206 | 0.18760226 | 0.00526166 | 0.04205674 |
| PPP1R14D | 0.71560356 | 1.05808588 | 0.34248232 | 0.00451683 | 0.04212374 |
| CD160 | 0.11136823 | 0.45802503 | 0.3466568 | 0.00487601 | 0.04320267 |
| S100A16 | 0.43528428 | 0.82513788 | 0.3898536 | 0.00534994 | 0.04339295 |
| CAPN3 | 0.40043038 | 0.11818662 | −0.2822438 | 0.00759541 | 0.04376152 |
| PCDH7 | 0.1952414 | 0.40640019 | 0.21115879 | 0.0064664 | 0.04490177 |
| IGSF9 | −0.2474561 | 0.20973574 | 0.45719184 | 0.00617357 | 0.04514562 |
| PRELP | 0.62414135 | 0.78269456 | 0.15855321 | 0.00613684 | 0.04620602 |
| SCGN | 0.33016884 | 0.60556595 | 0.27539711 | 0.00549141 | 0.04748045 |
| FABP2 | 0.41986091 | 0.92152928 | 0.50166837 | 0.00550865 | 0.04775468 |
| DPT | 0.63062493 | 0.80924138 | 0.17861646 | 0.00538954 | 0.04857466 |
| CXCL11 | 1.84925282 | 1.34285142 | −0.5064014 | 0.00491767 | 0.04929774 |

TABLE 4

| ID | Description | Set Size | Enrichment | NES | P value | p. adjust | qvalues | rank | Leading edge | Core enrichment | Core enrichment |
|---|---|---|---|---|---|---|---|---|---|---|---|
| WP3865 | Novel | 19 | −0.743728404 | −2.037620585 | 2.04E−05 | 0.005510615 | 0.004327467 | 399 | tags = 68%, | 57506/841/7186/8772/ | MAVS/CASP8/TRAF2/FADD/ |
| | intracellular | | | | | | | | list = 14%, | 7187/7124/8517/4790/ | TRAF3/TNF/IKBKG/NFKB1/ |
| | components | | | | | | | | signal = 59% | 843/64343/3627/7706/ | CASP10/AZI2/CXCL10/TRIM25/ |
| | of RIG-I-like | | | | | | | | | 23586 | DDX58 |
| | receptor pathway | | | | | | | | | | |
| WP4666 | Hepatitis B | 55 | −0.551231028 | −1.900840881 | 3.50E−05 | 0.005510615 | 0.004327467 | 950 | tags = 62%, | 2353/4318/5601/2033/ | FOS/MMP9/MAPK9/EP300/ |
| | infection | | | | | | | | list = 33%, | 1026/842/5604/581/ | CDKN1A/CASP9/MAP2K1/BAX/ |
| | | | | | | | | | signal = 42% | 637/10971/1386/4772/ | BID/YWHAQ/ATF2/NFATC1/ |
| | | | | | | | | | | 10000/4087/5608/596/ | AKT3/SMAD2/MAP2K6/BCL2/ |
| | | | | | | | | | | 57506/841/4088/3654/ | MAVS/CASP8/SMAD3/IRAK1/ |
| | | | | | | | | | | 8772/7187/7124/23118/ | FADD/TRAF3/TNF/TAB2/ |
| | | | | | | | | | | 836/51135/6714/6773/ | CASP3/IRAK4/SRC/STAT2/ |
| | | | | | | | | | | 6777/8517/4790/843/ | STAT5B/IKBKG/NFKB1/CASP10/ |
| | | | | | | | | | | 208/23586 | AKT2/DDX58 |
| WP437 | EGF/EGFR | 43 | −0.595876575 | −1.955761507 | 6.03E−05 | 0.006335484 | 0.004975234 | 521 | tags = 49%, | 10617/163/1759/6812/ | STAMBP/AP2B1/DNM1/STXBP1/ |
| | signaling | | | | | | | | list = 18%, | 5037/9138/3635/3636/ | PEBP1/ARHGEF1/INPP5D/INPPL1/ |
| | pathway | | | | | | | | signal = 41% | 4846/1399/8440/5728/ | NOS3/CRKL/NCK2/PTEN/ |
| | | | | | | | | | | 9046/9101/2308/1950/ | DOK2/USP8/FOXO1/EGF/ |
| | | | | | | | | | | 10253/6714/10451/6777/ | SPRY2/SRC/VAV3/STAT5B/ |
| | | | | | | | | | | 382 | ARF6 |
| WP231 | TNF-alpha | 32 | −0.636949119 | −1.968362076 | 0.000102578 | 0.008078056 | 0.006343669 | 907 | tags = 78%, | 56957/5601/1457/842/ | OTUD7B/MAPK9/CSNK2A1/CASP9/ |
| | signaling | | | | | | | | list = 31%, | 581/4794/637/329/ | BAX/NFKBIE/BID/BIRC2/ |
| | pathway | | | | | | | | signal = 54% | 7295/598/10010/5608/ | TXN/BCL2L1/TANK/MAP2K6/ |
| | | | | | | | | | | 56616/841/7186/8772/ | DIABLO/CASP8/TRAF2/FADD/ |
| | | | | | | | | | | 4217/840/7133/7124/ | MAP3K5/CASP7/TNFRSF1B/TNF/ |
| | | | | | | | | | | 23118/836/11140/8517/ | TAB2/CASP3/CDC37/IKBKG/ |
| | | | | | | | | | | 4790 | NFKB1 |
| WP254 | Apoptosis | 39 | −0.58043486 | −1.872641175 | 0.000190486 | 0.012000609 | 0.009424037 | 853 | tags = 67%, | 8738/842/835/665/ | CRADD/CASP9/CASP2/BNIP3L/ |
| | | | | | | | | | list = 29%, | 581/4794/637/7157/ | BAX/NFKBIE/BID/TP53/ |
| | | | | | | | | | signal = 48% | 329/598/596/56616/ | BIRC2/BCL2L1/BCL2/DIABLO/ |
| | | | | | | | | | | 1676/331/841/7186/ | DFFA/XIAP/CASP8/TRAF2/ |
| | | | | | | | | | | 8772/840/7133/7187/ | FADD/CASP7/TNFRSF1B/TRAF3/ |
| | | | | | | | | | | 7124/836/8517/4790/ | TNF/CASP3/IKBKG/NFKB1/ |
| | | | | | | | | | | 843/834 | CASP10/CASP1 |
| WP4658 | Small cell | 33 | −0.596472495 | −1.847121195 | 0.000376733 | 0.018685152 | 0.014673386 | 838 | tags = 67%, | 1026/842/581/3910/ | CDKN1A/CASP9/BAX/LAMA4/ |
| | lung cancer | | | | | | | | list = 29%, | 637/7157/2272/1282/ | BID/TP53/FHIT/COL4A1/ |
| | | | | | | | | | signal = 48% | 329/10000/598/596/ | BIRC2/AKT3/BCL2L1/BCL2/ |
| | | | | | | | | | | 841/7186/5728/7709/ | CASP8/TRAF2/PTEN/ZBTB17/ |
| | | | | | | | | | | 7187/836/8517/4790/ | TRAF3/CASP3/IKBKG/NFKB1/ |
| | | | | | | | | | | 208/4149 | AKT2/MAX |
| WP1772 | Apoptosis | 41 | −0.562788485 | −1.837888127 | 0.000471095 | 0.018685152 | 0.014673386 | 853 | tags = 63%, | 8738/842/835/581/ | CRADD/CASP9/CASP2/BAX/ |
| | modulation and | | | | | | | | list = 29%, | 637/7157/329/27429/ | BID/TP53/BIRC2/HTRA2/ |
| | signaling | | | | | | | | signal = 45% | 598/596/56616/1676/ | BCL2L1/BCL2/DIABLO/DFFA/ |
| | | | | | | | | | | 331/841/3654/8772/ | XIAP/CASP8/IRAK1/FADD/ |
| | | | | | | | | | | 4217/840/7133/7187/ | MAP3K5/CASP7/TNFRSF1B/TRAF3/ |
| | | | | | | | | | | 3303/836/9131/4790/ | HSPA1A/CASP3/AIFM1/NFKB1/ |
| | | | | | | | | | | 843/834 | CASP10/CASP1 |
| WP4880 | Host-pathogen | 10 | −0.799895633 | −1.848436462 | 0.000474544 | 0.018685152 | 0.014673386 | 399 | tags = 70%, | 57506/7187/6773/8517/ | MAVS/TRAF3/STAT2/IKBKG/ |
| | interaction | | | | | | | | list = 14%, | 4790/23586/5610 | NFKB1/DDX58/EIF2AK2 |
| | of human | | | | | | | | signal = 61% | | |
| | coronaviruses - | | | | | | | | | | |
| | interferon | | | | | | | | | | |
| | induction | | | | | | | | | | |
| WP4655 | Cytosolic | 18 | −0.679479882 | −1.843307529 | 0.000874815 | 0.030618536 | 0.024044631 | 493 | tags = 83%, | 6351/6352/3553/5435/ | CCL4/CCL5/IL1B/POLR2F/ |
| | DNA-sensing | | | | | | | | list = 17%, | 57506/841/8772/8517/ | MAVS/CASP8/FADD/IKBKG/ |
| | pathway | | | | | | | | signal = 70% | 4790/81030/843/834/ | NFKB1/ZBP1/CASP10/CASP1/ |
| | | | | | | | | | | 3627/7706/23586 | CXCL10/TRIM25/DDX58 |
| WP4329 | miRNA role in | 20 | −0.661109121 | −1.843807695 | 0.001014835 | 0.031967317 | 0.025103825 | 335 | tags = 40%, | 3654/7187/7124/23118/ | IRAK1/TRAF3/TNF/TAB2/ |
| | immune response | | | | | | | | list = 12%, | 51135/8517/4790/3586 | IRAK4/IKBKG/NFKB1/IL10 |
| | in sepsis | | | | | | | | signal = 36% | | |
| WP4868 | Type I interferon | 11 | −0.751780371 | −1.789804692 | 0.001417809 | 0.033873897 | 0.026601055 | 399 | tags = 55%, | 57506/7187/51135/6773/ | MAVS/TRAF3/IRAK4/STAT2/ |
| | induction and | | | | | | | | list = 14%, | 23586/5610 | DDX58/EIF2AK2 |
| | signaling during | | | | | | | | signal = 47% | | |
| | SARS-CoV-2 | | | | | | | | | | |
| | infection | | | | | | | | | | |
| WP3851 | TLR4 | 11 | −0.746699608 | −1.777708641 | 0.001609387 | 0.033873897 | 0.026601055 | 383 | tags = 73%, | 3635/3654/7187/7124/ | INPP5D/IRAK1/TRAF3/TNF/ |
| | signaling and | | | | | | | | list = 13%, | 23118/51135/8517/4790 | TAB2/IRAK4/IKBKG/NFKB1 |
| | tolerance | | | | | | | | signal = 63% | | |
| WP558 | Complement and | 43 | 0.493852855 | 1.859991035 | 0.001671108 | 0.033873897 | 0.026601055 | 822 | tags = 51%, | 2152/5327/7035/1675/ | F3/PLAT/TFPI/CFD/ |
| | coagulation | | | | | | | | list = 28%, | 717/2155/5627/3053/ | C2/F7/PROS1/SERPIND1/ |
| | cascades | | | | | | | | signal = 37% | 718/7056/2158/1604/ | C3/THBD/F9/CD55/ |
| | | | | | | | | | | 5624/3075/5104/2161/ | PROC/CFH/SERPINA5/F12/ |
| | | | | | | | | | | 5328/5329/5340/3426/ | PLAU/PLAUR/PLG/CFI/ |
| | | | | | | | | | | 730/629 | C7/CFB |
| WP1971 | Integrated | 16 | −0.674858539 | −1.78049312 | 0.001689113 | 0.033873897 | 0.026601055 | 887 | tags = 88%, | 983/1026/842/581/ | CDK1/CDKN1A/CASP9/BAX/ |
| | cancer | | | | | | | | list = 31%, | 7157/4087/596/11200/ | TP53/SMAD2/BCL2/CHEK2/ |
| | pathway | | | | | | | | signal = 61% | 841/4088/4217/571/ | CASP8/SMAD3/MAP3K5/BACH1/ |
| | | | | | | | | | | 5728/836 | PTEN/CASP3 |
| WP3858 | Toll-like | 11 | −0.745060431 | −1.773806162 | 0.001697974 | 0.033873897 | 0.026601055 | 335 | tags = 45%, | 3654/7187/51135/8517/ | IRAK1/TRAF3/IRAK4/IKBKG/ |
| | receptor | | | | | | | | list = 12%, | 4790 | NFKB1 |
| | signaling | | | | | | | | signal = 40% | | |
| | related to | | | | | | | | | | |
| | MyD88 | | | | | | | | | | |
| WP5108 | Familial | 12 | 0.725437495 | 1.923130668 | 0.001720579 | 0.033873897 | 0.026601055 | 576 | tags = 75%, | 51129/337/4023/4035/ | ANGPTL4/APOA4/LPL/LRP1/ |
| | hyperlipidemia | | | | | | | | list = 20%, | 338328/3949/5360/27329/ | GPIHBP1/LDLR/PLTP/ANGPTL3/ |
| | type 1 | | | | | | | | signal = 60% | 335 | APOA1 |
| WP481 | Insulin | 34 | −0.55468277 | −1.745853983 | 0.001974281 | 0.036582262 | 0.028727925 | 950 | tags = 68%, | 2353/6616/2309/5601/ | FOS/SNAP25/FOXO3/MAPK9/ |
| | signaling | | | | | | | | list = 33%, | 10580/6814/5604/1978/ | SORBS1/STXBP3/MAP2K1/EIF4EBP1/ |
| | | | | | | | | | signal = 46% | 5608/6810/6812/50488/ | MAP2K6/STX4/STXBP1/MINK1/ |
| | | | | | | | | | | 8773/3636/4217/11183/ | SNAP23/INPPL1/MAP3K5/MAP4K5/ |
| | | | | | | | | | | 5728/2308/1977/5770/ | PTEN/FOXO1/EIF4E/PTPN1/ |
| | | | | | | | | | | 382/2997/208 | ARF6/GYS1/AKT2 |

A recursive feature elimination procedure fitted Random Forest (RF) model recursively removed the weakest features until the specified number of features was reached in each random subsampling, to generate a matrix of Proteins×Selections. An RFE procedure was used to identify the relevant features to be used for generating a classifier (or ensemble of classifiers). The ‘caret’ package in R facilitates a process of backward selection where less important predictors are gradually eliminated based their importance ranking. This is determined by an external estimator. The RFE procedure may include four steps: 1). Ranking Features: the inventors ranked features based on their importance using the “rocc” model, incorporating repeated Cross-validation (CV); 2). Removing Redundant Features: Redundant features with correlation coefficient>0.7 were removed to mitigate multi-collinearity, achieved through the ‘findCorrelation’ function; 3). Prioritizing Protein Variables: the inventors employed the Random Forrest ‘rfFuncs’ model in conjunction with repeated CV within the ‘rfe’ function. This helped prioritize key protein variables, enhancing predictor selection for the inventors' analyses. 4. Integrating Protein Selection Matrix generated from RFE into a ranked protein list using R package “RobustRankAgg”.

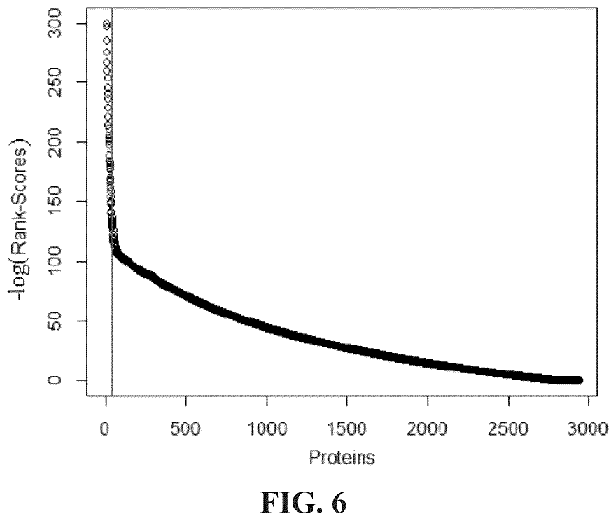

The inventors further integrated the Proteins×Selections matrix into ranking scores. The inventors plotted the ranking scores and set a cutoff criterion of −log(Rank-Score)>136 for proteomic classifier, as depicted in FIG. 6. The 37 proteins passing the criterion were designated as proteomic classifier-37 (PC37, as shown in Table 5, below).

TABLE 5

| Name | Score | log(Score) | inverse logScore |
|---|---|---|---|
| ADGRG1 | 0 | −300 | 300 |
| TRIM21 | 1.92E−315 | −300 | 300 |
| IL15 | 7.80E−298 | −297.10783 | 297.1078331 |
| MMP10 | 2.43E−285 | −284.61396 | 284.6139595 |
| SOST | 1.19E−275 | −274.92296 | 274.9229582 |
| SOD2 | 9.89E−268 | −267.00483 | 267.0048336 |
| POMC | 4.90E−261 | −260.31015 | 260.3101546 |
| KLRF1 | 3.08E−255 | −254.51096 | 254.5109599 |
| MMP12 | 1.31E−245 | −244.88307 | 244.8830692 |
| IL10 | 4.43E−241 | −240.35322 | 240.3532244 |
| CPM | 5.55E−237 | −236.2555 | 236.2554954 |
| BPIFB1 | 3.69E−229 | −228.43271 | 228.4327099 |
| GALNT5 | 6.92E−222 | −221.15986 | 221.1598614 |
| ITGB6 | 4.64E−215 | −214.33366 | 214.3336562 |
| CCDC80 | 3.49E−212 | −211.45777 | 211.4577746 |
| CEACAM5 | 5.99E−206 | −205.22257 | 205.2225694 |
| CDCP1 | 1.90E−203 | −202.72206 | 202.7220554 |
| POF1B | 4.32E−201 | −200.36455 | 200.3645504 |
| CAPS | 7.34E−199 | −198.13458 | 198.1345821 |
| EDIL3 | 4.34E−190 | −189.36298 | 189.3629836 |
| KDR | 6.53E−185 | −184.18511 | 184.1851141 |
| SELPLG | 4.70E−183 | −182.32758 | 182.3275806 |
| CLEC4G | 2.80E−181 | −180.55268 | 180.5526847 |
| CCL27 | 1.40E−179 | −178.85339 | 178.8533901 |
| DDC | 5.98E−178 | −177.22352 | 177.2235227 |
| KRT19 | 2.57E−170 | −169.59022 | 169.5902215 |
| FUT3_FUT5 | 7.65E−169 | −168.11656 | 168.1165586 |
| ROBO2 | 2.01E−167 | −166.69655 | 166.6965497 |
| CXCL14 | 3.87E−163 | −162.41203 | 162.41203 |
| KRT8 | 5.67E−159 | −158.2464 | 158.2463959 |
| PRSS8 | 6.42E−155 | −154.19218 | 154.1921769 |
| SCGB1A1 | 5.72E−151 | −150.24277 | 150.2427662 |
| AOC3 | 8.03E−150 | −149.09553 | 149.0955331 |
| AGR2 | 1.04E−148 | −147.98261 | 147.9826133 |
| WNT9A | 2.63E−142 | −141.58031 | 141.5803108 |
| IGFBPL1 | 2.79E−141 | −140.55498 | 140.5549811 |
| TNFRSF13B | 1.07E−137 | −136.96941 | 136.9694073 |

Gene Ontology analysis of PC37 revealed significant biological processes involved in bronchiole development, negative regulation of smooth muscle proliferation, and regulation of nonspecific immune response including interferon-alpha production and defense response to virus by host (Table 6).

TABLE 6
Gene Ontology Analysis of 37 proteomic classifier (PC37)

| GO ID | Gene Ontology Term | q-value | Protein Symbol |
|---|---|---|---|
| GO: 0010811 | Positive regulation of cell-substrate | 0.0093 | EDIL3, AGR2, CCDC80, KDR |
| | adhesion | | |
| GO: 0030889 | Negative regulation of B cell proliferation | 0.0078 | TNFRSF13B, IL10 |
| GO: 0032647 | Regulation of interferon-alpha production | 0.0022 | IL10, MMP12 |
| GO: 0034021 | Response to silicon dioxide | 0.0065 | SOD2, SCGB1A1 |
| GO: 0045214 | Sarcomere organization | 0.0052 | KRT8, KRT19 |
| GO: 0050691 | Regulation of defense response to virus by | 0.0058 | IL15, MMP12 |
| | host | | |
| GO: 0050777 | Negative regulation of immune response | 0.0041 | CLEC4G, IL10, MMP12, |
| | | | TRIM21 |
| GO: 0060435 | Bronchiole development | 0.0002 | ITGB6, MMP12 |
| GO: 0060706 | Cell differentiation involved in embryonic | 0.0005 | IL10, SOD2 |
| | placenta development | | |
| GO: 0070268 | cornification | 0.0093 | KRT8, MKRT19, PRSS8 |
| GO: 0140131 | Positive regulation of lymphocyte | 0.0039 | CCL27, CXCL14 |
| | chemotaxis | | |
| GO: 1903902 | Positive regulation of viral life cycle | 0.0039 | CLEC4G, TRIM21 |
| GO: 1904706 | Negative regulation of vascular associated | 0.0065 | IL10, SOD2 |
| | smooth muscle cell proliferation | | |

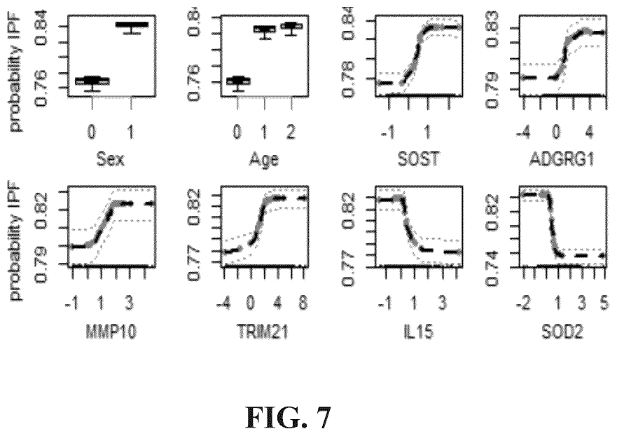

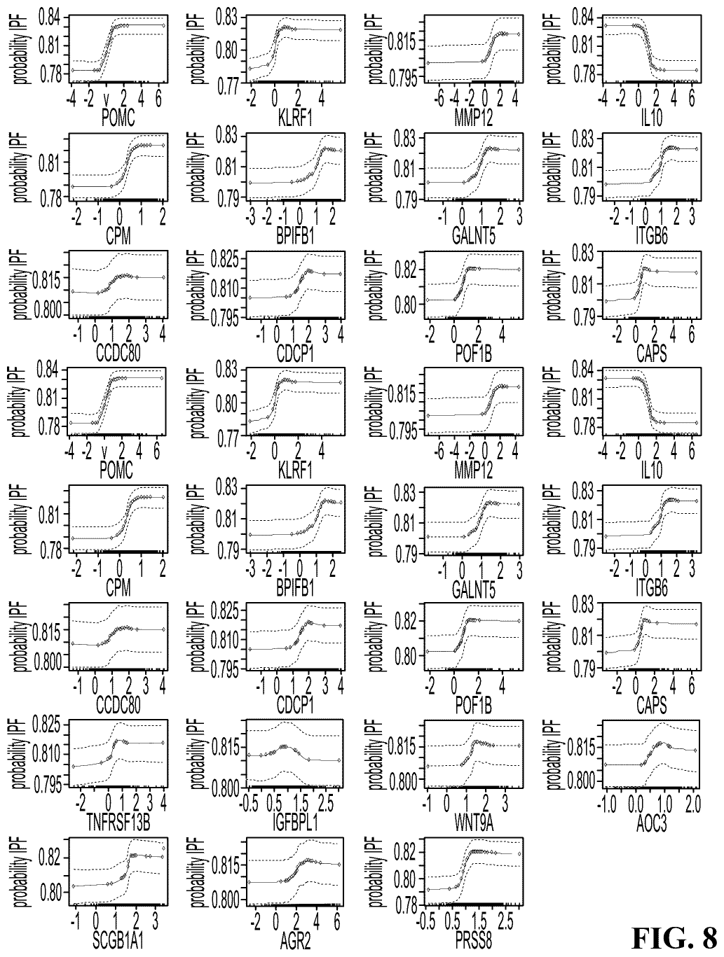

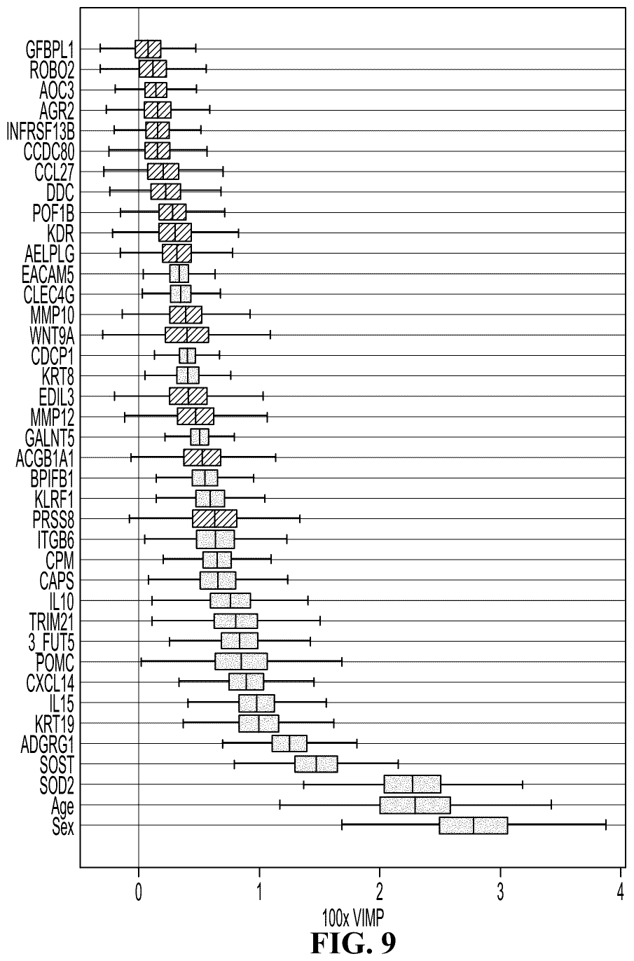

Partial effects of the PC37 features associated with IPF probability are displayed in FIG. 7 and FIG. 8. Increased abundance of interleukin-15 (IL15) and superoxide dismutase 2 (SOD2) were associated with CTD-ILD probabilities, while the abundance of the other features, sex and age scores were positively associated with IPF probabilities as shown in FIG. 7. The variable importance (VIMP) of PC37 with sex and age scores were ranked decreasingly from bottom to top in FIG. 9.

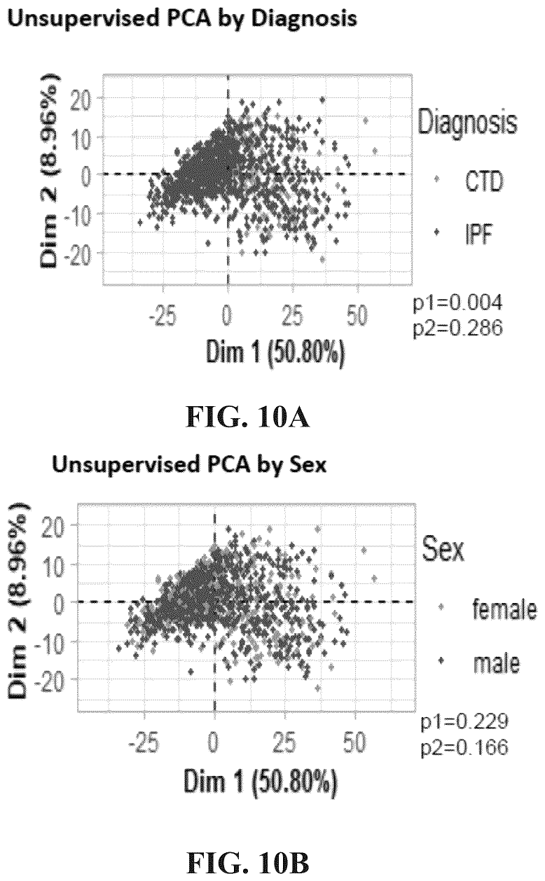

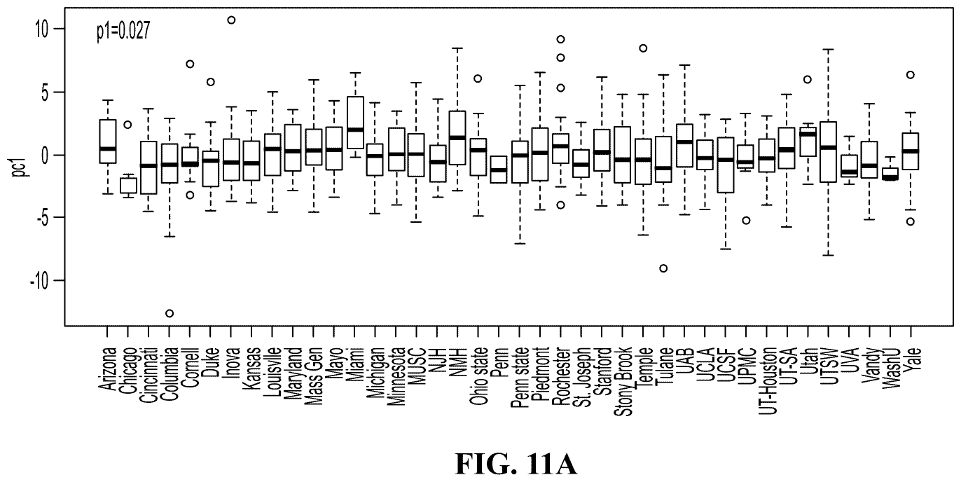

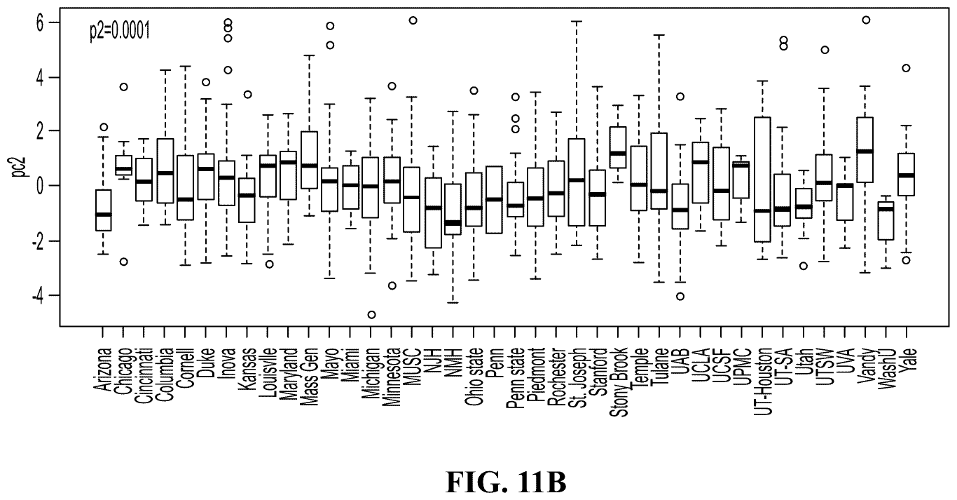

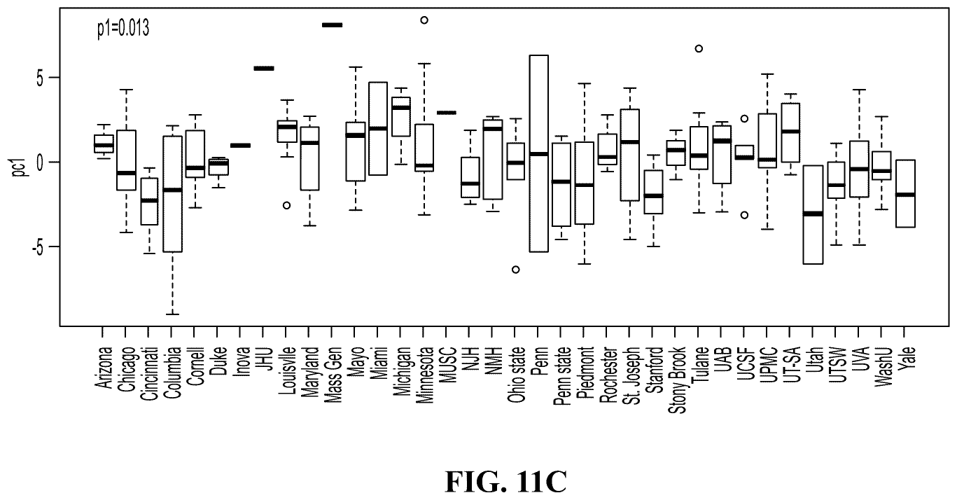

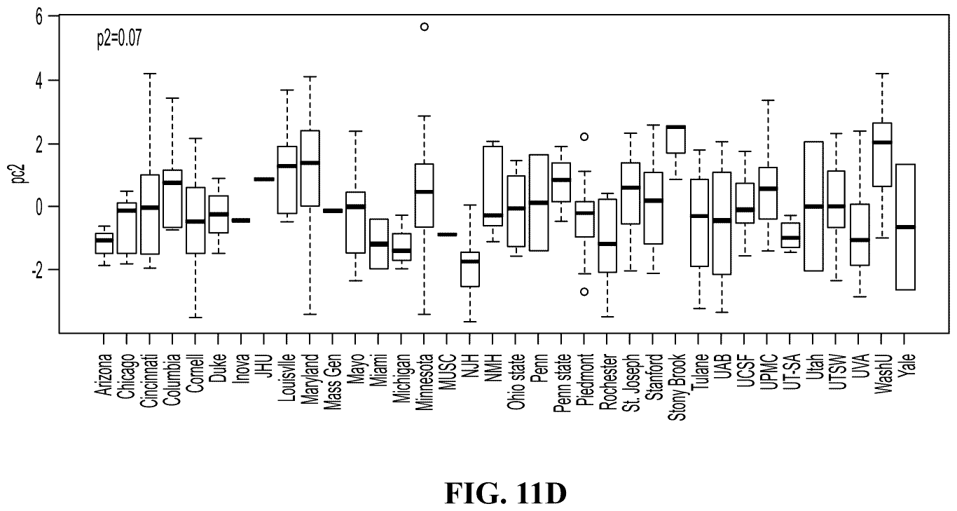

Unsupervised PCA of the training cohort demonstrated only mild separation between CTD-ILD and IPF in PC1, and not in PC2 (FIG. 10A), and no significant separation between male and female patients (FIG. 10B). Stratification of PFF samples by all 42 medical centers demonstrated extremely significant variations in both PC1 and PC2 with p<2e-16, while CTD-ILD samples only showed mild to moderate site variations. However, supervised PCA restricted to the PC37 markedly alleviated site-by-site variation (FIG. 11). PC37 showed similar alleviation in the test sample sites but couldn't palliate the difference between RECITAL with UC-Davis or other sites.

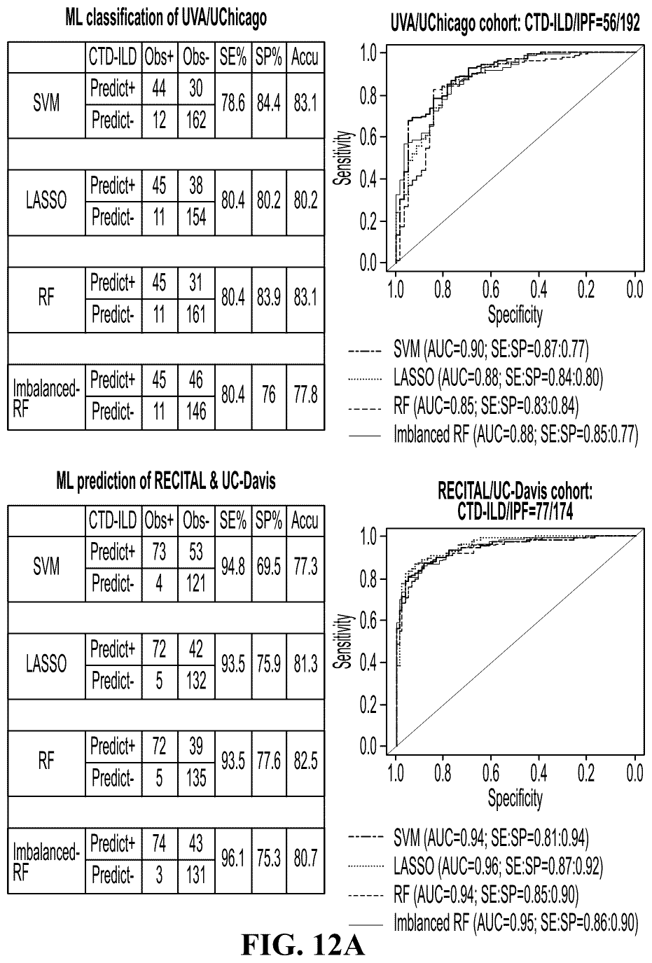

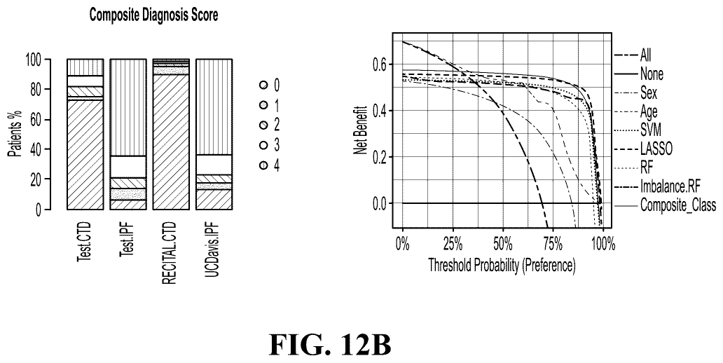

Use of PC37 with sex and age score in 4 machine learning models with different strengths and weakness, showed relatively equivalent performance in the test cohort, assuring generalizability and discriminative signal strength. The median of binary classification based on 100× random subsampling is summarized in FIG. 12. All three models displayed similar sensitivity (78.6%-80.4%) and specificity (76%-84.4%). ROC curve analysis using the continuous classification values confirmed the consistency of all models with AUC 0.85-0.90. Alternatively, the imbalanced-RF model in the PFF training cohort displayed slightly lower sensitivity (76.8%) and specificity (78.1%) in the test cohort, but similar AUC (0.88) for ROC curve analysis of the class probabilities.

For single-sample classification, the inventors repeated the 4 machine learning models validated by test cohort above in RECITAL CTD-ILD and UC-Davis IPF patients. Each case was classified iteratively using its own training cohorts. The median values of the binary classification values from 100× random subsampling of PFF training cohort are summarized in FIG. 12. Imbalanced-RF using PC37 with sex and age demonstrated comparable sensitivity and specificity with the other three machine learning models. ROC curve analysis of continuous classification values further confirmed consistency across models with AUC 0.94-0.96. Similarly, RECITAL CTD-ILD cases consistently separated from IPF in UVA/UChicago test cohort in all models (AUC=0.94-0.96), and UC-Davis IPF cases separated from CTD-ILD in UVA/UChicago test cohort (AUC=0.84-0.87). IPF probabilities of RF and imbalanced-RF models showed consistent distributions of CTD-ILD between RECITAL and UVA/UChicago CTD-ILD samples, or IPF between UC-Davis and UVA/UChicago IPF samples. These findings further affirm robustness of the inventors' approaches to differential diagnosis across machine learning models, independent of the existing site variation or technical batch-effect seen with RECITAL cases.

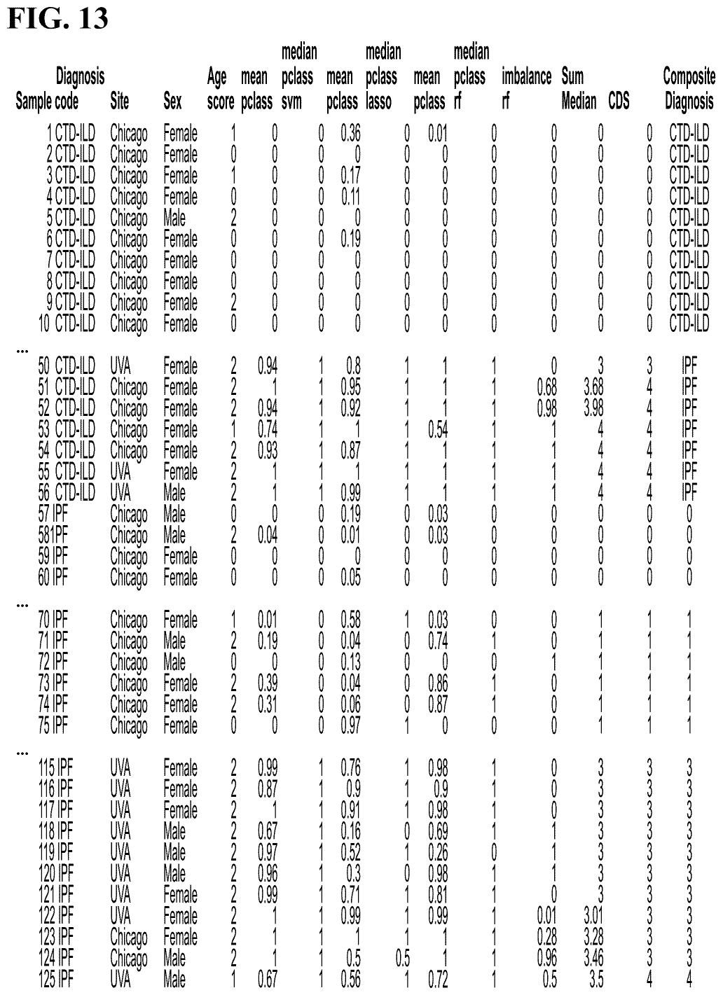

The inventors also computed a composite diagnosis score (CDS) for each sample (FIG. 12, 0-4 on the bottom left right panel). Specifically, 75% (42/56) of CTD-ILD and 79% (152/192) of IPF samples in the test cohort, 96% (74/77) of CTD-ILD in RECITAL, and 77% (134/174) of IPF in UC-Davis exhibited correct classification against clinical diagnosis (See FIG. 13 for a selection of example results). Overall, CDS analysis of 4 models confirmed 78.2% (194/248) accuracy in test cohort and 82.9% (208/251) in single-sample classification dataset.

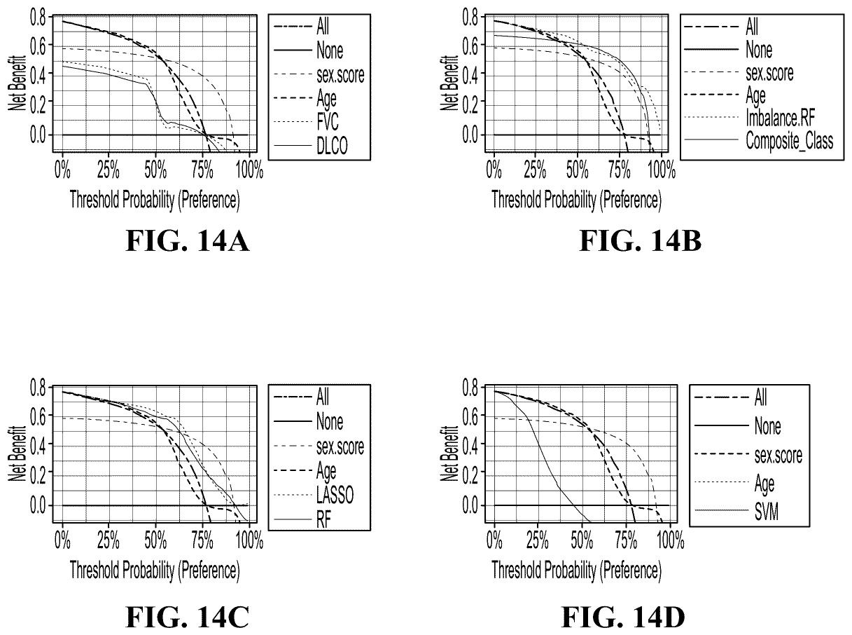

Referring to FIG. 14, decision cure analysis of test cohort confirmed sex and age as the most significant clinical parameters distinguishing CTD-ILD from IPF. Therefore, the inventors compared the decision curves between machine learning models with sex and age. Imbalance-RF and composite classification outperformed sex across the entire preference range, and age when >37.5% (FIG. 14B). LASSO regression and RF surpassed sex preference<62.5%, and age when >37.5% (FIG. 14C). SVM surpassed sex from 0%-18% preference (Figure E14D). In RECITAL/UC-Davis datasets, the machine learning models and composite classification surpassed sex across the entire preference range, and age when >50% in providing a net benefit of classification (See FIG. 12).

The inventors examined 10 false negative classifications in the UVA/UChicago test cohort. Despite 10 sub-categories of CTD-ILD, 6 of 10 false negative classifications by CDS occurred predominantly in RA-ILD cases. Five of 6 misclassifications in the 21 RA-ILD cases were over age 65 (Fisher exact test p=0.046).

This comprehensive study utilized proteomics and machine learning techniques to successfully develop and validate a proteomic classifier capable of distinguishing cases of CTD-ILD from IPF. The integration of various datasets allowed establishment of a robust framework for disease classification. Balancing the datasets through random subsampling, ensured an unbiased representation of cases with matched diagnosis and sex, allowing meaningful comparisons. The identified proteins and pathways demonstrate that aberrant immunity and fibrosis pathways are differentially activated in CTD-ILD versus IPF.

The machine learning-derived proteomic classification models exhibited high discriminatory power, with Harrell's C-statistic values ranging from 0.84 to 0.95 in both mixed test cohorts and the single-sample approach. The probabilities of each protein help establish protein characterization of each disease. Iterative classification of single-samples followed by composite scoring methods across all four machine learning models established a single-patient diagnosis model mimicking clinical practice settings. Performance of the classifier was similar to a whole transcriptome approach for the classification of UIP in transbronchial lung biopsies. However, a plasma-based classification offers an advantage in patients too fragile to undergo bronchoscopic or surgical lung biopsy. Further, decisional curve analyses demonstrate benefit both in diagnostic clarity and preference over sex, age and FVC and DLCO percentage predicted.

The “gold standard” diagnosis of IPF, requires exclusion of CTD-ILDs, based on clinical factors such as age and sex, rheumatologic signs and symptoms, and interpretation of serologies utilizing ACR criteria, in a MDD review. However, MDD itself can be error-prone, time consuming, and is limited to tertiary academic centers. Despite MDD, over a third of cases lack a confident diagnosis, and over 10% are misclassified with ongoing reclassification required. When considering discordance between the proteomic classifier and MDD, it is important to account for these limitations in the MDD. The systems and methods descried herein (e.g., using proteomic classifiers) offer a molecular characterization of cases that may not be classified by clinical criteria. Another possibility is that IPF may occur independent of and concurrent to CTD. Thus, it is contemplated that a proteomics' classification model could be developed with three output classes: IPF, CTD-ILD, and both.

Cohort comparisons showed that IPF cases were more often male, while a higher proportion of CTD-ILD patients identified as non-White race, consistent with prior studies. Difficulties making a definite diagnosis of CTD-ILD can result in low confidence diagnosis of IPF or the research designation of IPAF. This may result in gender and racial disparities, given that no clear treatment algorithm exists for the IPAF designation, as studies specifically addressing this population are lacking. Blood-based proteomics combined with machine learning can address these gaps in knowledge and provide an objective supplemental tool to the MDD diagnosis of ILD.

The two-group comparison revealed 88 significant proteins differentiating CTD-ILD from IPF. GSEA illustrates that the non-specific immune response and EGF/EGFR signaling pathways are enhanced in CTD-ILD when compared to IPF. Whereas, activated complement and coagulation cascades pathway demonstrated a stronger role in IPF than in CTD-ILD.

The 37-protein classifier results from variable importance ranking and multicollinearity control, followed by a backward selection of protein features to mitigate sex and site variations, underscoring its potential clinical relevance. Several examples are presented, showing their associated partial effects on the probability of having IPF. For instance, proteins like sclerostin (SOST), adhesion G protein-coupled receptor G1 (ADGRG1), matrix metalloproteinase 10 (MMP10), IL15, and SODS2 exhibit discernible associations with IPF probability. SOST functions to inhibit Wnt signaling pathway, a well-recognized pathway implicated in fibrosis. ADGRG1, aka GPR56, functions as a marker of cytotoxic T cells, which associate with risk for poor prognosis in IPF. TRIM21, also known as Ro52, is a major autoantigen in Sjogren's disease and systemic lupus erythematosus, and in the inventors' analysis, the partial effect favors higher levels in IPF and lower levels in CTD-ILD. Absence or deficiency of TRIM21 may in cases of CTD, alter the IRF4/5 axis to favor differentiation of antibody-secreting plasma cells.

Machine learning models have varying advantages and disadvantages related to their algorithms that can limit generalizability. Decision curve analysis demonstrated that different machine learning models surpassed sex and age at different range of threshold for clinical preference, illustrating the benefit of combining multiple models. SVM and RF are intuitively biased in modeling imbalanced data. To compensate, the inventors used random subsampling to balance both diagnostic class and sex ratio in the training cohort, which is an approach that would likely be beneficial even when performing the systems and methods herein for other disease state differentiations. SVM aims to find the optimal hyperplane that best separates classes, while RF is designed to reduce overfitting compared to single decision trees. However, both models can be sensitive to noisy data and outliers. Crucial sample filtering procedures identified and removed 25 technical outliers. In addition, LASSO regression does not naturally provide probabilities for each class. The inventors instead used linked values that give linear classifiers for downstream ROC analysis. Strong correlations among the selected features can cause overfitting of LASSO regression model. A step was introduced in feature selection to remove multicollinearity.

Proteomic misclassifications, although present, were comparable to the existing MDD-based approach. In RECITAL and UC-Davis cases, the misclassification rate against MDD was 12.7% (32/251) and the “unclassifiable” rate was 4.4% (11/251) for a combined 17.1%. This may indicate the inherent complexity of differentiating certain subcategories, particularly RA-ILD. Misclassified CTD-ILD cases from the UVA/UChicago cohort were mostly RA-ILD over age 65. The MUC5B promoter variant is more strongly associated with UIP phenotype in RA, suggesting shared genetic susceptibility with IPF. Several IPF associated protein markers such as MMP7 are known to differentiate RA from RA-ILD suggesting that perhaps some of these cases are RA with IPF, not RA-ILD resulting from RA.

Overall, the inventors' validation studies successfully demonstrated a blood-based protein classifier incorporating 37 proteins, sex, and age helping to better characterize protein differences between CTD-ILDs and IPF. The AUCs values were at a level commonly used in the clinical setting. Importantly, PC37 effectively alleviated site variation in both training and test cohorts. Despite heparin stored plasma in RECITAL leading to observed distinctions in supervised PCA, single-sample model using composite diagnosis score (CDS) confirmed an accuracy of 96% in identifying CTD-ILD cases, with scores of 3 or 4. While some variation in AUCs existed across all 4 models, use of a single-patient composite score enables more nuanced assessment for cases that may biologically reside on the spectrum between CDT-ILD and IPF.

Interpretation of functional pathways should be performed with caution given the small number of proteins in PC37 applied to attaining pathways. The Olink platform used in this investigation is semi-quantitative and therefore actual application in clinical practice would require conversion and confirmation of the data and the model to platforms easily executed across different clinical labs. Confirmation of performance of each protein with ELISA should be based on attaining the same antibody used in Olink assay, and likely explains variability.

### Contemplated Embodiments

The techniques, technologies, algorithms, and advantages described herein may be implemented in a variety of practical applications, which may serve to improve systems and methods used or performed by several different individuals, companies, and/or institutions involved in healthcare decision making.

In one category of embodiments, systems and methods may be configured to function as a tool to improve how diagnostically-relevant information can be provided to and used by clinicians and other healthcare professionals to differentiate between similarly-presenting diseases like ILD-related disorders. For example, a user interface may be provided which can receive (via user input or accessing data from an EMR or other medical record) patient-specific demographic data, test results, and/or a clinician's proposed possible disease states. (In some instances, the proposed possible disease states may be fewer than the number of target disease states for which a model/ensemble was trained—such as if the clinician has already ruled out one or more of the possible target disease states—in which case systems and methods may re-train or fine tune the model/ensemble according to some or all of the steps of FIG. 4.) The systems and methods may then utilize this information to perform a process such as in FIG. 1, to differentiate among the possible disease states from which the healthcare team cannot confidently reach a diagnosis using other methods.

Alternatively or additionally, the systems and methods serving as differentiation aids may output a report or other indication to the clinician, which may include: a suggestion of which types of data should be collected via which types of testing, patient examination, or patient history that will be most likely to improve differential diagnosis confidence (e.g., based on a ranking of features, feature pairs, or correlations from an RFE or similar process), including an ordering of tests to be performed based on settings that take into account patient comfort and disruption, invasiveness, and cost; an indication (with or without confidence level) of which of the set of possible disease states is likely present; an indication or explanation of which data points and feature correlations for the given patient provided the most discriminatory confidence underlying the tool's indication of which disease state is likely present.

In other examples, the systems and methods described herein can be utilized to guide diagnosis processes when healthcare teams have difficulty in differentiating among similarly-presenting diseases. These implementations may apply the algorithms and processes described above in specific care management platforms to promote and/or balance a number of factors, such as: reducing time to final differential diagnosis and commencement of treatment; reducing or managing the number of clinician specialties that may be required or become involved in differential diagnosis for patients; reducing the number of lab or imaging tests, or guiding the sequence of such tests, to promote efficiency (whether in terms of cost, number of tests, or patient comfort). For example, the processes and innovations described above could be integrated into an Electronic Medical Records (EMR) or Electronic Health Records (EHR) system, for management and access to patient data, document encounters, to enable a clinical decision support module within the EMR. Such embodiments could implement a variety of notifications within the clinician portal/user interface to recommend specific tests, by utilizing existing information regarding the patient (e.g., demographic, radiology, serologic, examination, etc.) to assess which currently-unknown data features would provide the best discriminatory value (in the absolute sense, or relative to cost and patient comfort) using for example the ranked feature lists obtained through process 400 or other RFE/alternative approaches, and which tests could best provide that information. For example, after a clinician enters information indicative of a category or group of potential similarly-presenting disease states (by entering symptoms into a given patient's EMR that are reflective of a category of similarly-presenting disease states (e.g., ILD-related symptoms), entering one or more diagnosis codes, or specifying that the patient likely has one of a set of possible diseases), the system could analyze what current information is available for that patient that has been determined to be relevant to discriminating among the possible disease states and then recommend the next best type of test to perform to obtain diagnostically-relevant information (such as suggesting a test for IL-15 protein levels or flag CTD-ILD vs IPF as a differential diagnosis that should be made). In other examples, a standalone platform for advanced diagnostic support could be utilized independent of an EMR/EHR. The platform could include a clinician-facing user interface that might include visualization tools like biomarker trends or decision trees, ranking of features determined to be relevant to differential diagnosis, and/or explanations of diagnostic reasonings.

In further examples, systems and methods of the present disclosure may be integrated into a laboratory testing platform. Thus, a clinician's initial diagnosis of a class of possible disorders (e.g., ILD-related diseases) could trigger a protein or biomarker test order that is provided to the laboratory testing platform. The systems and methods could determine which specific data (e.g. protein counts, correlations, etc.) should be detected in testing of a sample to be provided and/or emphasized in the report to be returned to the clinician. In some circumstances, where a given lab does not have a particularized test for the requested biomarkers, the systems and methods may instead suggest a set of standard tests or panels which in combination can provide the clinician with results that will provide the best differential diagnosis confidence level. In further examples, a laboratory testing platform which receives a request for a given type of serological, pathological, or histological test that is customarily used to diagnose a specific type of disorder (e.g., IPF) that is known to be of a set of similarly-presenting disorders (e.g., ILD-related disorders), the laboratory testing platform may suggest or automatically process a request for a related test that can help confirm differential diagnosis as among the set of similarly-presenting disorders.

In further examples, systems and methods of the present disclosure may be utilized in clinical decision support tools which may be combined or integrated with payer modules or risk management modules. For example, some EMR/EHR platforms may include a payer integration module that can interface with payer or risk management systems to check coverage for a given prescribed test, obtain preauthorization, and flag whether a given test requires additional prior testing or analysis. In the case of ILD-related disorders, as an example, if an ordered test would be specific to one or a few disorders out of a larger set of similarly-presenting disorders, these systems and methods may flag that another test could be conducted which would be approved and provide a better differential diagnosis as between ILD-related disorders. Or, if a prescription is entered for a therapy specific to a given class of ILD-related disorders, but the payer integration module detects that a differential diagnosis was not yet done or sufficient test results and other features were not yet entered into the EMR to allow for such a differential diagnosis (e.g., ruling out IPF, if the therapy is meant for CTD-ILD), the system may require such differential diagnosis be confirmed prior to authorization for the prescribed therapy. Likewise, a risk management system may be integrated with a clinical decision support tool that flags or suggests alternative or additional tests before a clinician proceeds with action based on an assumption of IPF vs CTD-ILD (or other similarly-presenting disorders).

In the foregoing specification, implementations of the disclosure have been described with reference to specific example implementations thereof. It will be evident that various modifications may be made thereto without departing from the broader spirit and scope of implementations of the disclosures as set forth in the following claims. The specification and drawings are, accordingly, to be regarding in an illustrative sense rather than a restrictive sense.

What is claimed is:

1. A method for distinguishing among similarly-presenting lung diseases, comprising:
obtaining a preliminary diagnosis of a category of similarly-presenting potential lung diseases;
obtaining a first data set corresponding to protein counts found in a blood sample from a patient;
obtaining a second data set corresponding to additional data regarding the patient;
providing an indication of the preliminary diagnosis, the first data set, and the second data set to a trained machine learning model;
determining a predicted differential diagnosis of a given lung disease of the category of similarly-presenting potential lung diseases, based upon an output of the trained machine learning model;
outputting a recommended treatment using the predicted differential diagnosis; and
obtaining confirmation of the predicted differential diagnosis and the recommended treatment.

2. The method of claim 1, wherein the second data set comprises at least one of: an age of the patient, a sex of the patient, or a race of the patient.

3. The method of claim 1, further comprising entering a background monitoring state, the background monitoring state comprising:
monitoring a patient database for a new data set;
rerunning the machine learning model using the new data set;
obtaining an updated diagnosis;
alerting a clinician if the updated diagnosis differs from the predicted differential diagnosis; and
storing an anonymized data set based on the new data set if the updated diagnosis matches the predicted differential diagnosis.

4. The method of claim 3, wherein the new data set comprises at least one of: an updated protein count, an age of the patient, a sex of the patient, a race of the patient, or a symptom experienced by the patient.

5. The method of claim 1, wherein the trained machine learning model was trained by:
obtaining a set of disease state classes belonging to the category of similarly-presenting potential lung diseases;
obtaining a training dataset of patient records in which all of the patient records include a confirmed diagnosis of one of the set of disease state classes and no patient records correspond to patients who had none of the set of disease state classes;
determining features of the training dataset that are relevant to differential diagnoses as among the set of disease state classes;
eliminating features of at least a portion of the training dataset that may be relevant to diagnosis of one or more of the disease state classes, but are not relevant to differential diagnosis as between the disease state classes, to create a reduced training dataset; and
training at least one machine learning model using the reduced training dataset to create the trained machine learning model.

6. A system for classifying among a defined set of similarly-presenting diseases, the system comprising:
a communication interface,
an electronic processor, and
a non-transitory computer-readable medium storing software instructions, which, when executed by the electronic processor, cause the electronic processor to:
receive a user input indicating a preliminary diagnosis from a clinician of a set of possible diseases for a given patient;
obtain a data set corresponding to circulating blood protein data of the given patient;
provide the data set to a trained machine learning model;
determine a predicted diagnosis from the set of similar diseases;
output a recommended treatment using the predicted diagnosis; and
obtain confirmation of the predicted diagnosis and the recommended treatment.
